(19) United States
(12) Patent Application Publication
(10) Pub. No.: US 2026/0337268 A1
(43) Pub. Date: Oct. 29, 2026

(54) METHOD OF TREATING CANCER WITH AN ANTI TMEFF2XCD3 BISPECIFIC ANTIBODY

(71) Applicant: Janssen Biotech, Inc., Horsham, PA (US)

(72) Inventors: Anjali Avadhani, Rockville, MD (US); Subhasree Basu, Hatfield, PA (US); Pharavee Jaiprasart, North Wales, PA (US); Hong Xie, Dresher, PA (US); David Yao, Villanova, PA (US)

(21) Appl. No.: 19/109,886

(22) PCT Filed: Sep. 6, 2023
(86) PCT No.: PCT/IB2023/058820

§ 371 Date: Mar. 7, 2025

Related U.S. Application Data
(60) Provisional application No. 63/405,188, filed on Sep. 9, 2022.
(60) Provisional application No. 63/420,146, filed on Oct. 28, 2022.

(51) Int. Cl. C07K 16/28; A61K 9/00; A61K 39/00; A61P 35/04

(52) U.S. Cl. CPC C07K 16/2809; A61K 9/0019; A61P 35/04; A61K 2039/505; C07K 2317/31; C07K 2317/55; C07K 2317/565

(57) ABSTRACT

Methods of treating a cancer in a patient comprising administering an anti TMEFF2×CD3 bispecific antibody as disclosed herein.

### CROSS-REFERENCE TO RELATED APPLICATION

This application claims priority to U.S. Ser. No. 63/405,188 filed Sep. 9, 2022 and to U.S. Ser. No. 63/420,146 filed Oct. 28, 2022, the disclosure of each of which is incorporated by reference herein in its entirety.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Jul. 7, 2023, is named JBI6759WOPCT1_SL and is 32,707 bytes in size.

### BACKGROUND

Prostate cancer is the second most common cancer in men worldwide, and the sixth leading cause of cancer-related death. Globally, there are approximately 1,100,000 new cases and 300,000 mortalities every year, comprising 4 percent of all cancer deaths. It is estimated that 1 in every 6 men will be diagnosed with the disease during his lifetime. Prostate cancer risk strongly correlates with age: around three-quarters of cases occur in men over 65 years old with the largest number of cases in those aged 70-74. It is estimated from post-mortem data that around a half of men in their fifties and 80% of men aged 80 have histological evidence of cancer in the prostate. At the early stages, the 5-year survival rate nears 100%. When the cancer has metastasized, however, the 5-year survival rate drops to 28%, and there remains a need for effective treatments for advanced-stage prostate cancer.

TMEFF2 is a conserved cell-membrane-bound proteoglycan also known as TENB2, HPP1, or tomoregulin-2. It is a 41 kDa transmembrane protein that consists of 2 follistatin-like domains (FS1 and FS2) and 1 epidermal growth factor (EGF)-like domain.

TMEFF2 expression is retained throughout all stages of disease in prostate cancer, with limited expression in extra-prostatic tissues. Direct correlation between increasing TMEFF2 expression levels and high-grade tumor strongly suggests that TMEFF2 is associated with disease progression, and possible androgen independence, in the advanced prostate cancer disease setting (Afar D E, Bhaskar V, Ibsen E, et al. Preclinical validation of anti-TMEFF2-auristatin E-conjugated antibodies in the treatment of prostate cancer. Mol Cancer Ther. 2004; 3(8): 921-932). TMEFF2 RNA and protein expression is observed in both the brain and prostate. Low levels transcript and protein expression are detected in retina and conjunctival epithelium, and transcript only without detected protein in nonpigmented ciliary body epithelium in the eye and ganglion cells in the myenteric plexus of colon.

Current treatments for prostate cancer include surgery, radiation and hormone therapies. Treatment aimed at eradicating the tumor is unsuccessful in 30% of men, who develop recurrent disease that is usually manifest first as a rise in plasma prostate-specific antigen (PSA) followed by spread to distant sites. Because prostate cancer cells depend on the androgen receptor (AR) for their proliferation and survival, men with advanced prostate cancer are treated with agents that block the production of testosterone (e.g., GnRH agonists), alone or in combination with anti-androgens (e.g., bicalutamide), which antagonize the effect of any residual testosterone on AR. These treatments reduce serum testosterone to castrate levels, which generally slows disease progression for a period of time. However, androgen depletion is usually effective for a limited duration and prostate cancer evolves to regain the ability to grow despite low levels of circulating androgens. Thus, most patients eventually succumb to cancer regrowth.

The need for more improved treatment and effective therapies for patients with advanced prostate cancer and whose disease has proved resistant to current therapy remains high.

### SUMMARY OF INVENTION

Provided herein are methods of treating cancer in a subject, comprising administering to the subject at least one dose of an anti TMEFF2×CD3 bispecific antibody of the disclosure.

In some embodiments the cancer is metastatic castration-resistant prostate cancer (mCRPC), including those with adenocarcinoma with small-cell or neuroendocrine features.

In some embodiments mCRPC subjects have received prior treatment with at least 1 prior novel AR-targeted therapy or chemotherapy.

In some embodiments, the administration is subcutaneous and the dose is from about 0.3 mg to about 6.0 mg of an anti TMEFF2×CD3 bispecific antibody of the disclosure.

In some embodiments, the administration is subcutaneous and the dose is 0.3 mg, 1.0 mg, 1.5 mg, 2.0 mg, 3.0 mg, 4.0 mg, or 6.0 mg of an anti TMEFF2×CD3 bispecific antibody of the disclosure.

In some embodiments, the administration is subcutaneous and the dose is given once weekly (Q1W).

In some embodiments, the administration is subcutaneous and the dose is given every two weeks weekly (Q2W).

In some embodiments, the administration is subcutaneous and the dose is from about 0.3 mg to about 6.0 mg and is given Q1W.

In some embodiments, the administration is subcutaneous and the dose is 0.3 mg, 1.0 mg, 1.5 mg, 2.0 mg, 3.0 mg, 4.0 mg, or 6.0 mg and is given Q1W.

In some embodiments, the administration is subcutaneous and the dose is 0.3 mg, 1.0 mg, 1.5 mg, 3.0 mg, or 6.0 mg and is given Q1W.

In some embodiments, the administration is subcutaneous and the dose is from about 0.3 mg to about 6.0 mg and is given Q2W.

In some embodiments, the administration is subcutaneous and the dose is 0.3 mg, 1.0 mg, 1.5 mg, 2.0 mg, 3.0 mg, 4.0 mg, or 6.0 mg and is given Q2W.

In some embodiments, the administration is subcutaneous and the dose is 2.0 mg, 3.0 mg, 4.0 mg, or 6.0 mg and is given Q2W.

In some embodiments, the methods of the disclosure comprise more than one iteration of the administration step.

In some embodiments, prior to the first dose of an anti TMEFF2×CD3 bispecific antibody of the disclosure, corticosteroid, antihistamine, and antipyretic premedication is administered to minimize the risks associated with cytokine release syndrome (CRS) and infusion-related reactions (IRR). The premedication dose(s) or schedule(s) is or can be reduced or omitted for subsequent doses. For subjects who experience a Grade 2 or higher CRS or IRR, pretreatment with corticosteroid is or can be administered for at least 1 subsequent dose administered to that subject.

In some embodiments, the anti TMEFF2×CD3 antibody administered in the disclosed methods of treatment comprises a first binding domain that binds TMEFF2 and a second binding domain that binds CD3, wherein (a) the first binding domain that binds TMEFF2 comprises the HCDRs of the VH having the amino acid sequence of SEQ ID NO: 13, and the LCDRs of the VL having the amino acid sequence of SEQ ID NO: 14, and the second binding domain that binds CD3 comprises the HCDRs of the VH having the amino acid sequence of SEQ ID NO: 17, and the LCDRs of the VL having the amino acid sequence of SEQ ID NO: 18; (b) the first binding domain that binds TMEFF2 comprises a HCDR1, HCDR2, HCDR3, LCDR1, LCDR2 and LCDR3 of SEQ ID NO 1, 2, 3, 4, 5 and 6, respectively, and the second binding domain that binds CD3 comprises a HCDR1, HCDR2, HCDR3, LCDR1, LCDR2 and LCDR3 of SEQ ID NO 7, 8, 9, 10, 11 and 12, respectively; (c) the first binding domain that binds TMEFF2 comprises a VH and VL of SEQ ID NO: 13 and 14, respectively, and the second binding domain that binds CD3 comprises a VH and VL of SEQ ID NO: 17 and 18, respectively; and/or (d) the first binding domain that binds TMEFF2 comprises a HC1 and LC1 of SEQ ID NO: 15 and 16, respectively; and the second binding domain that binds CD3 comprises a HC2 and LC2 of SEQ ID NO: 19 and 20 respectively.

### BRIEF DESCRIPTION OF THE DRAWINGS

FIG. 1 shows a schematic overview of the Study.

FIG. 2 shows a PK model predicted mean study drug serum concentration in human following a single IV at 4 u/kg or SC administration at 300 μg assuming median body weight of 75 kg. Abbreviations: F=bioavailability; IV=intravenous, MABEL=minimum anticipated biologic effect level, SC=subcutaneous

FIG. 3 shows Mean Serum Concentration-time curves of the study drug following the first SC injection of the study drug. Abbreviations: n=total number of subjects in each cohort; SC=subcutaneous.

FIG. 4 shows mean serum concentration-time curves of the study drug following Q1W SC dosing. Abbreviations: n=total number of subjects in each cohort; SC-subcutaneous.

FIG. 5 shows mean serum concentration-time curves of the study drug following Q2W SC dosing. Abbreviations: n=total number of subjects in each cohort; SC=subcutaneous.

FIG. 6 shows trends of subjects with lower body weight having higher exposures when compared to subjects with high body weight.

FIG. 7A shows the relationship between study drug volume distribution and body weight. FIG. 7B shows the relationship between study drug clearance and body weight.

FIG. 8 shows a waterfall plot of maximum percent reduction from baseline in PSA. Abbreviations: PSA=prostate-specific antigen; Q1W=once weekly; Q2W=every 2 weeks; SC=subcutaneous. The reference lines represent 30% and 50% decrease. Increase greater than 100% was set to 100%.

FIG. 9 shows a waterfall plot of maximum percent reduction from baseline in sum of diameters of target lesions. Abbreviations: Q1W=once weekly; Q2W=every 2 weeks; SC=subcutaneous; SoD=Sum of Diameters. The reference line represents 30% decrease. Increase greater than 100% was set to 100%.

FIG. 10 shows study drug concentration following different dosing regimens.

FIG. 11A and FIG. 11B show the effects of study drug dosing regimens on CD8+ T cells infiltration.

FIG. 12A and FIG. 12B show the effects of study drug dosing regimens on CD4+ T cells infiltration.

FIG. 13A-13F show the effects of study drug dosing regimens on T cell activation and proliferation. FIG. 13A-13C are CD4+ Prostate Infiltration Lymphocyte (PILs) cells activation markers and FIG. 13D-13F are CD8+ Prostate Infiltration Lymphocyte (PILs) cells activation markers.

FIG. 14A-14F show the effects of study drug dosing regimens on suppressive T cell markers. FIG. 14A-14C are CD4+ Prostate Infiltration Lymphocyte (PILs) cells activation markers and FIG. 14D-14F are CD8+ Prostate Infiltration Lymphocyte (PILs) cells activation markers.

FIG. 15A-15C show the effects of study drug dosing regimens on suppressive T cell markers. FIG. 15A-15B are CD4+ Prostate Infiltration Lymphocyte (PILs) cells activation markers and FIG. 15C are CD8+ Prostate Infiltration Lymphocyte (PILs) cells activation markers.

FIG. 16A-16B show the effects of study drug dosing regimens on myeloid cell infiltration.

FIG. 17A-17C show the effects of study drug dosing regimens on myeloid cell infiltration.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the methods as described herein. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any sub combination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” is used to encompass variations of ±10% or less, variations of ±5% or less, variations of ±1% or less, variations of ±0.5% or less, or variations of =0.1% or less from the specified value

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed disclosure. Embodiments described in terms of the phrase “comprising” (or its equivalents) also provide as embodiments those independently described in terms of “consisting of” and “consisting essentially of.” Embodiments described in terms of the phrase “consisting essentially of” (or its equivalents) also provide as embodiments those independently described in terms of “consisting of.”

### Method of Treatment

Provided herein are methods of treating cancer in a subject, comprising administering to the subject at least one dose of an anti TMEFF2×CD3 bispecific antibody of the disclosure, wherein the dose is a safe and therapeutically effective amount of the said anti TMEFF2×CD3 antibody.

“Subject” or “Patient” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein. In some embodiments, the subject or patient is human. Particularly, the subject according to the invention is a human. In some embodiments, the subject according to the invention has or is susceptible to have prostate cancer.

“Treat”, “treating” or “treatment” a subject with a pathologic condition such as cancer refers to an effect that alleviates the condition by killing the cancerous cells, but also refers to accomplishing one or more of the following: reducing the severity and/or duration of the disorder, delaying the progression of the disorder, slowing the progression of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder. Treatment as a prophylactic measure (i.e., prophylaxis) is also included.

As used herein, the terms “delaying the progression of” or “slowing the progression of” shall include (a) delaying or slowing the development of one or more symptoms or complications of the disease, condition or disorder; (b) delaying or slowing the development of one or more new/additional symptoms or complications of the disease, condition or disorder; and/or (c) delaying or slowing the progression of the disease, condition or disorder to a later stage or more serious form of said disease, condition or disorder.

As used in accordance with the present disclosure, the term “treating” means treating a mammal having prostate cancer by providing an effective amount of an anti TMEFF2×CD3 bispecific antibody with the purpose of reducing or eradicating cancer cells and/or prolonging survival of the mammal.

As used herein, the terms “effective amount” and “therapeutically effective amount” refers to an amount sufficient to achieve a concentration of compound which is capable of preventing or slowing down the disease to be treated. Such concentrations can be routinely determined by those of skilled in the art. The amount of the polypeptide actually administered will typically be determined by a physician or a veterinarian, in the light of the relevant circumstances, including the condition to be treated, the chosen route of administration, the actual compound administered, the age, weight, and response of the patient, the severity of the subject's symptoms, and the like. It will also be appreciated by those of skilled in the art that the dosage may be dependent on the stability of the administered antibody.

A therapeutically effective amount may vary depending on factors such as the disease state, age, sex, and weight of the individual, the physical condition of the patient, the duration of the treatment, the nature of concurrent therapy (if any), the specific formulations employed, the structure of the compounds or its derivatives, and the ability of a therapeutic or a combination of therapeutics to elicit a desired response in the individual. A therapeutically effective dose of the administered antibody polypeptide also depends on the type and severity of the cancer being treated, and the route of administration of the antibody polypeptide or the pharmaceutical composition of the antibody polypeptide.

In some embodiments, subjects to be treated include those who have metastatic castration-resistant prostate cancer (mCRPC), including adenocarcinoma with small-cell or neuroendocrine features.

In some embodiments, the treatment efficiency for prostate cancer can be assessed using CT scan, MRI or whole body PET scan or by measuring PSA level.

In some embodiments, the treatment efficiency for prostate cancer can be assessed by measuring the progression of soft tissue lesions by CT or MRI using the RECIST criteria. As used herein, the term “Response Evaluation Criteria In Solid Tumors (RECIST)” refers to a set of published rules that define when cancer patients improve (“respond”), stay the same (“stable”) or worsen (“progression”) during treatments. The original criteria were published in February 2000 by an international collaboration including the European Organization for Research and Treatment of Cancer (EORTC), National Cancer Institute (NCI) of the United States and the National Cancer Institute of Canada Clinical Trials Group. RECIST 1.1, published in January 2009, is an update to the original criteria. Usually, the skilled in the art concludes that the disease progresses (and hence that the patient is or is become resistant to a treatment) when at least a 20% increase in the sum of the longest diameter of target lesions, taking as reference the smallest sum longest diameter recorder since the treatment started or the appearance of one or more new lesions) by conventional methods of imaging such as computed tomography (CT).

In some embodiments, the treatment efficiency of prostate cancer can be assessed according to PCWG3 (Prostate Cancer Working Group 3) criteria.

The term “safe,” as it relates to a dose, dosage regimen, or method of treatment with an anti TMEFF2×CD3 bispecific antibody of the present disclosure, refers to a relatively low or reduced frequency and/or low or reduced severity of treatment-emergent adverse events (referred to as AEs or TEAEs) from the clinical trials conducted, e.g., Phase 1 clinical trials, compared to the standard of care or to another comparator. An adverse event is an untoward medical occurrence in a patient who was administered a medicinal product including adverse vital signs (heart rate, systolic and diastolic blood pressure, body temperature), adverse standard clinical laboratory tests (hematology, clinical chemistry, urinalysis, lipids, coagulation), allergic reactions/hypersensitivity, adverse local injection site reactions, or adverse EKG. In particular, safe as it relates to a dose, dosage regimen or treatment with an anti TMEFF2×CD3 bispecific antibody of the present disclosure refers to a relatively low or reduced frequency and/or low or reduced severity of adverse events associated with the administration of the antibody if attribution is considered to be possible, probable, or very likely due to the use of the anti TMEFF2×CD3 bispecific antibody.

### Route of Administration

The methods of the invention may comprise any administration means that achieve the intended purpose. Any suitable route of administration can be used to administer the antibody polypeptide or the pharmaceutical composition used in the method of treatment of the disclosure. For example, administration may be achieved by a number of different routes including, but not limited to, subcutaneous routes.

### Prostate Cancer

The disclosed methods can treat any cancer associated with TMEFF2. Exemplary cancer associated with TMEFF2 is prostate cancer. The term “cancer” as used herein refers to an abnormal growth of cells which tend to proliferate in an uncontrolled way and, in some cases, to metastasize (spread).

In some embodiments the methods of the disclosure can treat or slow the progression of prostate cancer.

The term “prostate cancer” as used herein refers to histologically or cytologically confirmed adenocarcinoma of the prostate and to neuroendocrine prostate cancer, a late manifestation of prostate adenocarcinoma and a hormone-refractory subtype of prostate cancer resulting from prostate cancer treatment. The course of prostate cancer from diagnosis to death is best categorized as a series of clinical states based on the extent of disease, hormonal status, and absence or presence of detectable metastases: localized disease, rising levels of prostate-specific antigen (PSA) after radiation therapy or surgery with no detectable metastases, and clinical metastases in the non-castrate or castrate state.

In the early stages of prostate cancer, the cancer is localized to the prostate. In these early stages, treatment typically involves either surgical removal of the prostate or radiation therapy to the prostate or observation only with no active intervention therapy in some patients. In the early stages where the prostate cancer is localized and requires intervention, surgery or radiation therapy are curative by eradicating the cancerous cells. However, about 30% of the time these procedures fail, and the prostate cancer continues to progress, as typically evidenced by a rising PSA level. Thus, a significant proportion of patients treated through surgery, radiation, or a combination of both, have recurrent disease, which can lead to the development of metastases, especially in the high-risk group—a transition to the lethal phenotype of the disease. Men whose prostate cancer has progressed following these early treatment strategies are said to have advanced or recurrent prostate cancer.

The term “locally advanced prostate cancer”, “advanced prostate cancer” or “advanced disease” refers to prostate cancers which have extended through the prostate capsule where all actively cancerous cells appear to be confined to the prostate and the associated organs or neighbor organs (e.g., seminal vesicle, bladder neck, and rectal wall). Advanced prostate cancer includes stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease, and these patients have substantially less favorable outcomes compared to patients having clinically localized (organ-confined) prostate cancer. Locally advanced disease is clinically identified by palpable evidence of induration beyond the lateral border of the prostate, or asymmetry or induration above the prostate base. Locally advanced prostate cancer is presently diagnosed pathologically following radical prostatectomy if the tumor invades or penetrates the prostatic capsule, extends into the surgical margin, or invades the seminal vesicles.

The term “high-risk localized prostate cancer” refers to locally advanced prostate cancer that has a probability of developing metastases or recurrent disease after primary therapy with curative intent. In some embodiments, high risk for development of metastases is defined as Prostate Specific Antigen Doubling Time (PSADT)<12 months, or <11 months, <10 months, <9 months, <8 months, <7 months, <6 months, <5 months, <4 months, <3 months, <2 months, or <1 month. In some embodiments, high risk for development of metastases is defined as prostate specific antigen doubling time (PSADT)<10 months. In some embodiments, high risk for development of metastases is defined as having a high Gleason score or bulky tumor.

The terms “metastatic prostate cancer” and “metastatic disease” mean prostate cancers which have spread to regional lymph nodes or to distant sites, and are meant to include stage D disease under the AUA system and stage T×N×M+ under the TNM system. As is the case with locally advanced prostate cancer, surgery is generally not indicated for patients with metastatic disease, and hormonal (androgen ablation) therapy or androgen-deprivation therapy (ADT) is the preferred treatment modality. Patients with metastatic prostate cancer eventually develop an androgen-refractory state within 12 to 18 months of treatment initiation, and approximately half of these patients die within 6 months thereafter. The most common site for prostate cancer metastasis is bone. Prostate cancer bone metastases are, on balance, characteristically osteoblastic rather than osteolytic (i.e., resulting in net bone formation). Bone metastases are found most frequently in the spine, followed by the femur, pelvis, rib cage, skull and humerus. Other common sites for metastasis include lymph nodes, lung, liver and brain. Metastatic prostate cancer is typically diagnosed by open or laparoscopic pelvic lymphadenectomy, whole body radionuclide scans, skeletal radiography, and/or bone lesion biopsy.

Androgen receptor (AR) is a member of the steroid and nuclear receptor superfamily and its function is regulated by the binding of androgens. AR is mainly expressed in androgen target tissues, such as the prostate, skeletal muscle, liver, and central nervous system (CNS), with the highest expression level observed in the prostate, adrenal gland, and epididymis. AR can be activated by the binding of endogenous androgens, including testosterone and 5-dihydrotestosterone (5a-DHT). Upon activation by androgens, AR mediates transcription of target genes that modulate growth and differentiation of prostate epithelial cells. AR signaling is crucial for the development and maintenance of male reproductive organs including the prostate gland, as genetic males harboring loss of function AR mutations and mice engineered with AR defects do not develop prostates or prostate cancer. This dependence of prostate cells on AR signaling continues even upon neoplastic transformation.

The term “androgen-deprivation therapy (ADT)” refers to the reduction of androgen levels in a prostate cancer patient to castrated levels of testosterone (<50 ng/dL). ADT includes surgical castration (orchiectomy) and/or the administration of gonadotropin-releasing hormone (also known as luteinizing hormone-releasing hormone [“LHRH”] agonists or antagonists to a human. Examples of LHRH agonists include, but are not limited to, goserelin acetate, histrelin acetate, leuprolide acetate, and triptorelin palmoate.

Anti-androgens are useful for the treatment of prostate cancer during its early stages. These treatments reduce serum testosterone to castrate levels, which generally slows disease progression for a period of time. However, prostate cancer often advances to a ‘hormone-refractory’ state in which the disease progresses in the presence of continued androgen ablation or anti-androgen therapy and most patients eventually succumb to cancer regrowth. Instances of antiandrogen withdrawal syndrome have also been reported after prolonged treatment with anti-androgens. Molecular profiling studies of castration-resistance prostate cancers commonly show increased androgen receptor (AR) expression, which can occur through AR gene amplification or other mechanisms.

The term “castration-sensitive prostate cancer” refers to cancer that is responsive to androgen-deprivation therapy (ADT) either as localized disease, biochemical relapse or in the metastatic setting. Castration-sensitive prostate cancer is categorized as non-metastatic or metastatic, depending on whether or not the prostate cancer has metastasized to other parts of the body.

The term “metastatic castration-sensitive prostate cancer” refers to cancer that has spread (metastasized) to other areas of the body, e.g., the bone, lymph nodes or other parts of the body in a male, and that is responsive to androgen-deprivation therapy (ADT).

The term “non-metastatic castration-sensitive prostate cancer” refers to cancer that has not spread (metastasized) in a male, and that is responsive to androgen-deprivation therapy (ADT). In some embodiments, non-metastatic castration-sensitive prostate cancer is assessed with bone scan and computed tomography (CT), magnetic resonance imaging (MRI) scans, or positron emission tomography (PET).

The term “CRPC” or “castration-resistant prostate cancer “refers” to a prostate cancer that continues to grow despite the suppression of male hormones that fuel the growth of prostate cancer cells. Castration-resistant prostate cancer (CRPC) is categorized as non-metastatic or metastatic, depending on whether or not the prostate cancer has metastasized to other parts of the body. Castration resistant prostate cancer (CRPC) is a lethal phenotype and almost all of patients will die from prostate cancer. Interestingly, while a small minority of CRPC does bypass the requirement for AR signaling, the vast majority of CRPC, though frequently termed “androgen independent prostate cancer” or “hormone refractory prostate cancer,” retains its lineage dependence on AR signaling. The term “metastatic castration-resistant prostate cancer” or mCRPC refers to castration-resistant prostate cancer that has metastasized to other parts of the human body.

The term “NM-CRPC” or “non-metastatic castration-resistant prostate cancer” refers to cancer that has not spread (metastasized) in a male, and that is resistant to androgen-deprivation therapy (ADT) i.e., that continues to grow despite the suppression of male hormones. In some embodiments, non-metastatic castration-sensitive prostate cancer is assessed with bone scan and computed tomography (CT), magnetic resonance imaging (MRI) scans or positron emission tomography (PET).

In some embodiments, the non-metastatic castration-resistant prostate cancer is a high-risk non-metastatic castration-resistant prostate cancer. The term “high risk nm-CRPC” refers to probability of a man with nm-CRPC developing metastases. In some embodiments, high risk for development of metastases is defined as prostate specific antigen doubling time (PSADT)<10 months, <9 months, <8 months, <7 months, <6 months, <5 months, <4 months, <3 months, <2 months, or <1 month. In some embodiments, high risk for development of metastases is defined as having local-regional recurrence (e.g. primary tumor bed, bladder neck, anastomotic area, pelvic lymph nodes).

The term “chemotherapy naive metastatic castration-resistant prostate cancer” refers to metastatic castration-resistant prostate cancer that has not been previously treated with a chemotherapeutic agent.

The term “neuroendocrine prostate cancer (NEPC)” also called treatment-related NEPC (tNEPC) is an aggressive androgen independent variant of prostate cancer that most commonly arises in later stages of mCRPC as a mechanism of treatment resistance (Wang H T et al, J. of Clinical Oncology, 2014, Vol 32, Issue 30, 3383-3390). This tumor does not secrete prostate-specific antient (PSA) and is a highly aggressive subtype of prostate cancer characterized by the following clinical features: unresponsiveness to hormone therapy, presence of lytic bone lesions, rapid disease progression, presence of visceral metastases, marked prostatic enlargement, and disproportionately low PSA levels in the setting of metastatic disease. Androgen receptor (AR) expression is typically low or absent in NEPC and the genes Aurora kinase A (AURKA) and N-Myc (MYCN) are frequently amplified.

The development of tNEPC is estimated to cause approximately 25% of the nearly 34,000 cases of lethal prostate cancer per year in the United States (A Jemal, F Bray, M M Center, et al: Global cancer statistics CA Cancer J Clin 61:69-90,2011). However, data from autopsy studies suggest that the incidence of NEPC may be significantly underestimated (P N Brawn, V O Speights: The dedifferentiation of metastatic prostate carcinoma Br J Cancer 59:85-88,1989). The amount of neuroendocrine differentiation increases with disease progression and correlates with patient exposure to long-term androgen-deprivation therapy. Preclinical studies also support the idea that transformation from PCa to tNEPC is promoted by androgen-deprivation therapy and may arise as a mechanism of resistance.

The present disclosure provides methods of treating or slowing the progression of prostate cancer in a subject by administering an anti TMEFF2×CD3 bispecific antibody. The methods of treating or slowing the progression of prostate cancer include but are not limited to treating castration-resistant prostate cancer (CRPC), metastatic castration-resistant prostate cancer (mCRPC), non-metastatic castration-resistant prostate cancer (NM-CRPC), relapsed prostate cancer disease following androgen receptor (AR)-targeted therapy, locally advanced prostate cancer, high-risk localized prostate cancer, castration sensitive prostate cancer, non-metastatic castration-sensitive prostate cancer, metastatic castration-sensitive prostate cancer, chemotherapy naive metastatic castration-resistant prostate cancer, or neuroendocrine prostate cancer.

In some embodiments, the methods of treatment of the disclosure comprise methods of treating or slowing the progression of prostate cancer in a subject with an anti TMEFF2×CD3 bispecific antibody, wherein the prostate cancer is metastatic castration-resistant prostate cancer (mCRPC).

### Antibody

The methods of the disclosure are practiced by administering a bispecific antibody that specially binds to human TMEFF2 and to human CD3. The anti TMEFF2×CD3 bispecific antibodies are useful for the treatment of cancer associated with TMEFF2, such as metastatic castration-resistant prostate cancer (mCRPC).

“TMEFF2” refers to human transmembrane protein with EGF like and two follistatin like domains 2, also called tomoregulin 2. The amino acid sequence of the full length human TMEFF2 is shown in SEQ ID NO: 27. The extracellular domain of TMEFF2 is shown in SEQ ID NO: 28 and spans residues 40-320 of the full length TMEFF2. TMEFF2 extracellular domain harbors three distinct subdomains, the Kazal-like 1 (residues 85-137), the Kazal-like 2 (residues 176-229) and the EGF domain (residues 261-301). The TMEFF2 EGF domain is shown in SEQ ID NO: 29. The TMEFF2 “membrane proximal region” refers to the TMEFF2 region of SEQ ID NO: 21, which encompasses the EGF domain and the N—C-terminal linker regions (e.g. residues 230-320 of full length human TMEFF2 of SEQ ID NO: 27). All references to proteins, polypeptides and protein fragments herein are intended to refer to the human version of the respective protein, polypeptide or protein fragment unless explicitly specified as being from a non-human species. Thus, “TMEFF2” means human TMEFF2 unless specified as being from a non-human species, e.g., “mouse TMEFF2” or “monkey TMEFF2” etc.

### SEQ ID NO: 27 (Full Length Human TMEFF2)

MVLWESPRQCSSWTLCEGFCWLLLLPVMLLIVARPVKLAAFPTSLSDCQT
PTGWNCSGYDDRENDLFLCDTNTCKFDGECLRIGDTVTCVCQFKCNNDYV
PVCGSNGESYQNECYLRQAACKQQSEILVVSEGSCATDAGSGSGDGVHEG
SGETSQKETSTCDICQFGAECDEDAEDVWCVCNIDCSQTNFNPLCASDGK
SYDNACQIKEASCQKQEKIEVMSLGRCQDNTTTTTKSEDGHYARTDYAEN
ANKLEESAREHHIPCPEHYNGFCMHGKCEHSINMQEPSCRCDAGYTGQHC
EKKDYSVLYVVPGPVRFQYVLIAAVIGTIQIAVICVVVLCITRKCPRSNR
IHRQKQNTGHYSSDNTTRASTRLI

### SEQ ID NO: 28 (Extracellular Domain of Human TMEFF2)

FPTSLSDCQTPTGWNCSGYDDRENDLFLCDTNTCKFDGECLRIGDTVTCV
CQFKCNNDYVPVCGSNGESYQNECYLRQAACKQQSEILVVSEGSCATDAG
SGSGDGVHEGSGETSQKETSTCDICQFGAECDEDAEDVWCVCNIDCSQTN
FNPLCASDGKSYDNACQIKEASCQKQEKIEVMSLGRCQDNTTTTTKSEDG
HYARTDYAENANKLEESAREHHIPCPEHYNGFCMHGKCEHSINMQEPSCR
CDAGYTGQHCEKKDYSVLYVVPGPVRFQYV

### TMEFF2 EGF Domain SEQ ID NO: 29

HHIPCPEHYNGFCMHGKCEHSINMQEPSCRCDAGYTGQHCE

### TMEFF2 Membrane Proximal Region SEQ ID NO: 21

NTTTTTKSEDGHYARTDYAENANKLEESAREHHIPCPEHYNGFCMHGKCE
HSINMQEPSCRCDAGYTGQHCEKKDYSVLYVVPGPVRFQYV

“CD3” refers to an antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 22. The extracellular domain spans residues 23-126 of the full length CD3. All references to proteins, polypeptides and protein fragments herein are intended to refer to the human version of the respective protein, polypeptide or protein fragment unless explicitly specified as being from a non-human species. Thus, “CD3” means human CD3 unless specified as being from a non-human species, e.g., “mouse CD3” “monkey CD3,” etc.

### SEQ ID NO: 22 (Human CD3 Epsilon)

MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI

As used herein the term “Antibody” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, dimeric, tetrameric or multimeric antibodies, single chain antibodies, domain antibodies and any other modified configuration of the immunoglobulin molecule that comprises an antigen binding site of the required specificity. The term antibody includes full length antibodies, whole antibodies, intact antibodies, antibody fragments, antigen binding fragment and antigen binding domains.

In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to some embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

The term “variable region” or “variable domain” refers to the heavy or light chain domain that is involved in the binding of the antibody to the antigen. The variable domains of the heavy or light chain (VH and VL, respectively) comprise four framework regions (FR) and three complementarity determining regions (CDRs).

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132:211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196:901-17), IMGT (Lefranc et al. (2003) Dev Comp Immunol 27:55-77) and AbM (Martin and Thornton J Bmol Biol 263:800-15, 1996). The correspondence between the various delineations and variable region numbering is described (see e.g., Lefranc et al. (2003) Dev Comp Immunol 27:55-77; Honegger and Pluckthun (2001), J Mol Biol 309:657-70; International ImMunoGeneTics (IMGT) database; Web resources, http://www_imgt_org). Available programs such as abYsis by UCL Business PLC may be used to delineate CDRs. The terms “CDR”, “HCDR1”, “HCDR2”, “HCDR3”, “LCDR1”, “LCDR2” and “LCDR3” as used herein include CDRs defined by any of the methods described supra, Kabat, Chothia, IMGT or AbM, unless otherwise explicitly stated in the specification. Correspondence between the numbering system, including, for example, the Kabat numbering and the IMGT unique numbering system, is well known to one skilled in the art (see, e.g., Kabat, supra; Chothia, supra; Martin, supra; Lefranc et al., supra).

TABLE 1

| | IMGT | Kabat | AbM | Chothia |
|---|---|---|---|---|
| V_{H} CDR1 | 27-38 | 31-35 | 26-35 | 26-32 |
| V_{H} CDR2 | 56-65 | 50-65 | 50-58 | 53-55 |
| V_{H} CDR3 | 105-117 | 95-102 | 95-102 | 96-101 |
| V_{L} CDR1 | 27-38 | 24-34 | 24-34 | 26-32 |
| V_{L} CDR2 | 56-65 | 50-56 | 50-56 | 50-52 |
| V_{L} CDR3 | 105-117 | 89-97 | 89-97 | 91-96 |

“Specifically binds,” “specific binding,” “specifically binding” or “binds” refer to a proteinaceous molecule binding to an antigen or an epitope within the antigen with greater affinity than for other antigens. Typically, the proteinaceous molecule binds to the antigen or the epitope within the antigen with an equilibrium dissociation constant (K_{D}) of about 1×10⁻⁷ M or less, for example about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein). The term “K_{D}” refers to the dissociation constant, which is obtained from the ratio of K_{D} to Ka (i.e., Kd/Ka) and is expressed as a molar concentration (M). K_{D} values for antibodies can be determined using methods in the art in view of the present disclosure. For example, the K_{D} of an antibody can be determined by using surface plasmon resonance, such as by using a biosensor system, e.g., a Biacore® system, or by using bio-layer interferometry technology, such as an Octet RED96 system. The smaller the value of the K_{D} of an antibody, the higher affinity that the antibody binds to a target antigen.

As used herein, an antibody that “binds to TMEFF2” or that “specifically binds to TMEFF2” refers to an antibody that binds to TMEFF2, preferably human TMEFF2, with a K_{D} of 1×10⁻⁷ M or less, preferably 1×10 8 M or less, more preferably 5×10⁻⁹ M or less, 1×10⁻⁹ M or less, 5×10⁻¹⁰ M or less, or 1×10⁻¹⁰ M, 5×10⁻¹¹ M, 1×10⁻¹¹ M, 5×10⁻¹² M, or 1×10⁻¹² M or less.

As used herein, an antibody that “binds to CD3” or that “specifically binds to CD3” refers to an antibody that binds to CD3, preferably human CD3, with a K_{D} of 1×10⁻⁷ M or less, preferably 1×10 8 M or less, more preferably 5×10⁻⁹ M or less, 1×10⁻⁹ M or less, 5×10⁻¹⁰ M or less, or 1×10⁻¹⁰ M, 5×10⁻¹¹ M, 1×10⁻¹¹ M, 5×10⁻¹² M, or 1×10⁻¹² M or less.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Bispecific anti-TMEFF2/anti-CD3 antibody”, “TMEFF2/CD3 antibody”, “anti TMEFF2×CD3 bispecific antibody” and the like refer to an antibody that binds to TMEFF2 and CD3.

In some embodiments, the anti TMEFF2×CD3 bispecific antibody administered in the method of treatment include whole antibodies or full-length antibodies, Fv fragments, single chain scFv fragments (scFv), Fab, F(ab)₂, or single chain antibodies.

The terms “Full length antibodies”, “whole antibodies” and “intact antibodies” are used herein interchangeably to refer to an antibody having a structure similar to a native antibody. “Intact antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

As used herein, the term “Antibody fragment”, and “antigen binding fragment” refers to a molecule other than an intact antibody. Antigen binding fragments may be synthetic, enzymatically obtainable or genetically engineered polypeptides and include portions of an immunoglobulin that bind an antigen, such as a VH, a VL, a VH and a VL, a Fab, a Fab′, a F(ab′)₂, a Fd and a Fv fragments, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a domain antibody (dAb) consisting of one VH domain or one VL domain, a shark variable IgNAR domain, a camelized VH domain, a VHH domain, a minimal recognition unit consisting of the amino acid residues that mimic the CDRs of an antibody, such as a FR3-CDR3-FR4 portion, the HCDR1, the HCDR2 and/or the HCDR3 and the LCDR1, the LCDR2 and/or the LCDR3, an alternative scaffold that bind an antigen, a bivalent domain antibody, a multispecific protein comprising the antigen binding fragment or any other antibody fragment that binds to an antigen but does not comprise a complete antibody structure.

In some embodiments, the method of the disclosure is practiced by administering a full length TMEFF2×CD3 bispecific antibody comprised of two heavy chains (HC1 and HC2) and two light chains (LC1 and LC2).

In some embodiments, the method of the disclosure is practiced by administering a TMEFF2×CD3 bispecific antibody comprising a heavy chain (HC1), a light chain (LC) and a single chain Fv (scFV).

“Single chain Fv” or “scFv” are fusion proteins comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. A scFv may have the VL and VH variable regions in either order, e.g., with respect to the N-terminal and C-terminal ends of the polypeptide, the scFv may comprise VL-linker-VH or may comprise VH-linker-VL. An scFv can comprise a linker peptide, such as two to about eight glycine or other amino acid residues, which connects the variable region of the heavy chain and the variable region of the light chain.

In some embodiments, the anti TMEFF2×CD3 bispecific antibodies used in the method of treatment of the disclosure include chimeric, humanized or fully human antibodies that specifically bind to TMEFF2 and CD3.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both.

Typically, a “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

Transgenic animals, such as mice, rat or chicken carrying human immunoglobulin (Ig) loci in their genome may be used to generate the antibodies used in the method of the disclosure, and are described in for example U.S. Pat. No. 6,150,584, Int. Patent Publ. No. WO1999/45962, Int. Patent Publ. Nos. WO2002/066630, WO2002/43478, WO2002/043478 and WO1990/04036. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (World Wide Web: regeneron.com), Harbour Antibodies (World Wide Web: harbourantibodies.com), Open Monoclonal Technology, Inc. (OMT) (World Wide Web: omtinc.net), KyMab (World Wide Web: kymab.com), Trianni (World Wide Web: trianni.com) and Ablexis (World Wide Web: ablexis.com) can be engaged to provide human antibodies directed against a selected antigen.

The antibodies generated by immunizing non-human animals may be humanized using methods well known in the art. Generally, a humanized or engineered antibody has one or more amino acid residues from a source that is non-human, e.g., but not limited to, mouse, rat, rabbit, non-human primate or other mammal. Exemplary humanization techniques including selection of human acceptor frameworks include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs or a subset of CDR residues of parental antibodies are transferred onto human frameworks that may be selected based on their overall homology to the parental frameworks, based on similarity in CDR length, or canonical structure identity, or a combination thereof.

Humanized antigen binding domains may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs for example to improve affinity of the antigen binding domain.

The anti TMEFF2×CD3 bispecific antibody used in accordance with the present disclosure can be produced by recombinant means, including from mammalian cell or transgenic preparations, or can be purified from other biological sources, as described herein or as known in the art. Antibodies used in the method of the present disclosure can be produced by a cell line, a mixed cell line, an immortalized cell or clonal population of immortalized cells, as well known in the art. Cell lines can be engineered to express the antibodies of the disclosure and the antibody can be produced intracellularly, in the periplasmic space, or directly secreted into the medium.

Cell lysate or supernatant comprising the anti TMEFF2×CD3 bispecific antibody can be purified using, for example, hydroxylapatite chromatography, gel electrophoresis, dialysis, and affinity chromatography. Other techniques for protein purification such as fractionation on an ion-exchange column, ethanol precipitation, Reverse Phase HPLC, chromatography on silica), chromatography on an anion or cation exchange resin are also available.

### Method of Treatment

In some embodiments, the method of the disclosure is practiced by administering a TMEFF2×CD3 bispecific antibody comprising a first variable domain that specifically bind TMEFF2 and a second variable domain that specifically binds CD3, wherein the first variable domain that specifically binds TMEFF2 comprises a VH of SEQ ID NO: 13 and a VL of SEQ ID NO: 14; and the second variable domain that specifically binds CD3 comprises a VH of SEQ ID NO: 17 and a VL of SEQ ID NO: 18.

In some embodiments, the method of the disclosure is practiced by administering a TMEFF2×CD3 bispecific antibody comprising a first variable domain that specifically bind TMEFF2 and a second variable domain that specifically binds CD3, wherein the first variable domain that specifically binds TMEFF2 comprises a HCDR1, a HCDR2, a HCDR3, a LCDR1, a LCDR2 and a LCDR3 of SEQ ID NO: 1, 2, 3, 4, 5, and 6, respectively; and the second variable domain that specifically binds CD3 comprises a HCDR1, a HCDR2, a HCDR3, a LCDR1, a LCDR2 and a LCDR3 of SEQ ID NO: 7, 8, 9, 10, 11, and 12, respectively.

In some embodiments, the method of the disclosure is practiced by administering a TMEFF2×CD3 bispecific antibody comprising a first variable domain that specifically bind TMEFF2 and a second variable domain that specifically binds CD3, wherein the first variable domain that specifically binds TMEFF2 comprises a heavy chain (HC) of SEQ ID NO: 15 and a light chain (LC) of SEQ ID NO: 16; and the second variable domain that specifically binds CD3 comprises a heavy chain (HC) of SEQ ID NO: 19 and a light chain (LC) of SEQ ID NO: 20.

In some embodiments, the methods of the disclosure are practiced by administering an anti TMEFF2×CD3 bispecific antibody comprising a first binding domain that binds TMEFF2 and a second binding domain that binds CD3, wherein (a) the first binding domain that binds TMEFF2 comprises the HCDRs of the VH having the amino acid sequence of SEQ ID NO: 13, and the LCDRs of the VL having the amino acid sequence of SEQ ID NO: 14, and the second binding domain that binds CD3 comprises the HCDRs of the VH having the amino acid sequence of SEQ ID NO: 17, and the LCDRs of the VL having the amino acid sequence of SEQ ID NO: 18; (b) the first binding domain that binds TMEFF2 comprises a HCDR1, HCDR2, HCDR3, LCDR1, LCDR2 and LCDR3 of SEQ ID NO 1, 2, 3, 4, 5 and 6, respectively, and the second binding domain that binds CD3 comprises a HCDR1, HCDR2, HCDR3, LCDR1, LCDR2 and LCDR3 of SEQ ID NO 7, 8, 9, 10, 11 and 12, respectively; (c) the first binding domain that binds TMEFF2 comprises a VH and VL of SEQ ID NO: 13 and 14, respectively, and the second binding domain that binds CD3 comprises a VH and VL of SEQ ID NO: 17 and 18, respectively; and/or (d) the first binding domain that binds TMEFF2 comprises a HC1 and LC1 of SEQ ID NO: 15 and 16, respectively; and the second binding domain that binds CD3 comprises a HC2 and LC2 of SEQ ID NO: 19 and 20 respectively.

In some embodiment, the method of the disclosure induces T cell activation and proliferation in a target-specific manner with no significant activation or proliferation of T cells in the periphery.

In some embodiments, the method of the disclosure induces T cell activation and proliferation in a target-specific manner.

In some embodiments, the method of the disclosure induces infiltration of activated and proliferating T cells and inflammatory cells in the prostate.

In some embodiments, the method of the disclosure increases intra-prostatic T cell numbers.

In some embodiments, the method of the disclosure does not induce significant numbers of suppressive T cell markers (PD1+Ki-67− and CD25+FoxP3).

In some embodiments, the method of the disclosure induces myeloid cell infiltration.

### Compositions

The method of the disclosure is also practiced by administering a composition comprising an anti TMEFF2×CD3 bispecific antibody described herein.

In the methods of the disclosure, the antibody may also be administered as a pharmaceutical composition comprising a therapeutically effective amount of the anti TMEFF2×CD3 bispecific antibody and optionally a pharmaceutically acceptable carrier.

### EXAMPLES

The following examples are provided to further describe some of the embodiments disclosed herein. The examples are intended to illustrate, not to limit, the disclosed embodiments.

### Example 1. Generation and Description of the Study Drug

Exemplary TMEFF2×CD3 bispecific antibody Ab1 will be used as study drug in the examples.

Ab1 is an immunoglobulin (Ig) G4-proline, alanine, alanine (IgG4 PAA) DuoBody® bispecific antibody that simultaneously binds to the cluster of differentiation (CD) 3 receptor complex on T lymphocytes (T-cells) and to the transmembrane protein with epidermal growth factor-like and 2 follistatin-like domains (TMEFF2) on tumor cells. It is hypothesized that via this binding activity, the bispecific antibody mediates synapse formation between T-cells and TMEFF2 expressing cells, leading to T-cell activation and subsequent lysis of TMEFF2 positive cells by perforin and granzymes secreted by cytotoxic T-cells. Ab1 has reduced binding to fragment crystallizable (Fc)γ receptors due to the leucine to alanine mutations at positions 234 and 235 and reduced fragment antigen-binding arm exchange due to the serine to proline mutation at position 228.

Ab1 was developed to evaluate the therapeutic potential of targeting TMEFF2 for CD3-mediated T cell redirection. The bispecific antibody was generated by controlled fragment antigen binding (Fab) arm exchange from 2 antibodies: TMEB762 and CD3B376. TMEB762 is an anti-TMEFF2 antibody generated by immunizing OmniRats (OMT™). CD3B376 is an anti-CD3ε antibody generated by immunizing OmniRats (OMT™).

The generation of Ab1 has been described in U.S. Publication No. 16,417,889 published Nov. 28, 2019 entitled “Monospecific and multispecific anti-TMEFF2 antibodies and their uses” which is incorporated herein by reference in its entirety. Assays to evaluate the functional activities of Ab1 and structural properties such as, e.g., amino acid sequences are also described in detail in US Publication No. 16,417,889 incorporated herein by reference in its entirety.

The TMEFF2 binding domain of Ab1 (TMCB150) comprises the HCDR1 of amino acid sequence SYSMS (SEQ ID NO: 1), the HCDR2 of amino acid sequence VISGSGGFTDYADSVKG (SEQ ID NO: 2), and the HCDR3 of amino acid sequence MPLNSPHDY (SEQ ID NO: 3) and the LCDR1 of amino acid sequence RASQGIRNDLG (SEQ ID NO: 4), the LCDR2 of amino acid sequence AASSLQS (SEQ ID NO: 5), and the LCDR3 of amino acid sequence LQDYNYPLT (SEQ ID NO: 6) using the Kabat delineation.

The CD3 binding domain of Ab1 (CD3B376) comprises the HCDR1 of amino acid sequence NNNAAWS (SEQ ID NO: 7), the HCDR2 of amino acid sequence RTYYRSKWLYDYAVSVKS (SEQ ID NO: 8), and the HCDR3 of amino acid sequence GYSSSFDY (SEQ ID NO: 9) and the LCDR1 of amino acid sequence TGTSSNIGTYKFVS (SEQ ID NO: 10), the LCDR2 of amino acid sequence EVSKRPS (SEQ ID NO: 11), and the LCDR3 of amino acid sequence VSYAGSGTLL (SEQ ID NO: 12) using the Kabat delineation.

The VH VL, HC and LC sequences of Ab1 (TMCB150) TMEFF2 and CD3 binding domains are described below.

### VH, VL, HC, LC of Ab1 TMEFF2 Binding Domain

### VH Amino Acid Sequence of TMCB150 TMEFF2 Binding Domain; SEQ ID NO: 13

EVQLLESGGGLVQPGGSLRLSCAASGFTFSSYSMSWVRQAPGKGLEWVSV
ISGSGGFTDYADSVKGRFTISRDNSKNTLYLQMNSLRAEDTAVYYCARMP
LNSPHDYWGQGTLVTVSS

### VL Amino Acid Sequence of TMCB150 TMEFF2 Binding Domain; SEQ ID NO: 14

DIQMTQSPSSLSASVGDRVTITCRASQGIRNDLGWYQQKPGKAPKLLIYA
ASSLQSGVPSRFSGSGSGTDFTLTISSLQPEDFATYYCLQDYNYPLTFGG
GTKVEIK

### HC Amino Acid Sequence of TMCB150 TMEFF2 Binding Domain; SEQ ID NO: 15

EVQLLESGGGLVQPGGSLRLSCAASGFTFSSYSMSWVRQAPGKGLEWVSV
ISGSGGFTDYADSVKGRFTISRDNSKNTLYLQMNSLRAEDTAVYYCARMP
LNSPHDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEAAGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK

### LC Amino Acid Sequence of TMCB150 TMEFF2 Binding Domain; SEQ ID NO: 16

DIQMTQSPSSLSASVGDRVTITCRASQGIRNDLGWYQQKPGKAPKLLIYA
ASSLQSGVPSRFSGSGSGTDFTLTISSLQPEDFATYYCLQDYNYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### HC Nucleic Acid Sequence of TMCB150 TMEFF2 Binding Domain; SEQ ID NO: 23

GAGGTGCAGCTGCTGGAAAGCGGCGGAGGCCTGGTGCAGCCCGGAGGAAG
CCTGAGACTCAGCTGTGCCGCCAGCGGCTTCACCTTCAGCAGCTACAGCA
TGAGCTGGGTCAGGCAGGCCCCTGGCAAAGGACTGGAGTGGGTGAGCGTG
ATTAGCGGCAGCGGCGGCTTCACCGATTACGCCGACAGCGTGAAGGGCAG
GTTCACCATCAGCAGGGACAATAGCAAGAACACCCTGTACCTGCAGATGA
ACAGCCTGAGGGCCGAGGACACCGCCGTGTACTACTGCGCCAGGATGCCC
CTGAACAGCCCTCACGACTACTGGGGCCAGGGAACCCTGGTGACCGTGTC
CAGCGCTTCCACCAAGGGCCCATCCGTCTTCCCCCTGGCGCCCTGCTCCA
GGAGCACCTCCGAGAGCACAGCCGCCCTGGGCTGCCTGGTCAAGGACTAC
TTCCCCGAACCGGTGACGGTGTCGTGGAACTCAGGCGCCCTGACCAGCGG
CGTGCACACCTTCCCGGCTGTCCTACAGTCCTCAGGACTCTACTCCCTCA
GCAGCGTGGTGACCGTGCCCTCCAGCAGCTTGGGCACGAAAACCTACACT
TGCAACGTAGATCACAAGCCCAGCAACACCAAGGTGGACAAGAGAGTTGA
GTCCAAATATGGTCCCCCATGCCCACCATGCCCAGCACCTGAGGCCGCCG
GGGGACCATCAGTCTTCCTGTTCCCCCCAAAACCCAAGGACACTCTCATG
ATCTCCCGGACCCCTGAGGTCACGTGCGTGGTGGTGGACGTGAGCCAGGA
AGACCCCGAGGTCCAGTTCAACTGGTACGTGGATGGCGTGGAGGTGCATA
ATGCCAAGACAAAGCCGCGGGAGGAGCAGTTCAACAGCACGTACCGTGTG
GTCAGCGTCCTCACCGTCCTGCACCAGGACTGGCTGAACGGCAAGGAGTA
CAAGTGCAAGGTCTCCAACAAAGGCCTCCCGTCCTCCATCGAGAAAACCA
TCTCCAAAGCCAAAGGGCAGCCCCGAGAGCCACAGGTGTACACCCTGCCC
CCATCCCAGGAGGAGATGACCAAGAACCAGGTCAGCCTGACCTGCCTGGT
CAAAGGCTTCTACCCCAGCGACATCGCCGTGGAGTGGGAGAGCAATGGGC
AGCCGGAGAACAACTACAAGACCACGCCTCCCGTGCTGGACTCCGACGGC
TCCTTCTTCCTCTACAGCAGGCTAACCGTGGACAAGAGCAGATGGCAGGA
GGGGAATGTCTTCTCATGCTCCGTGATGCATGAGGCTCTGCACAACCACT
ACACACAGAAGAGCCTCTCCCTGTCTCTGGGTAAA

### LC Nucleotide Sequence of TMCB150 TMEFF2 Binding Domain; SEQ ID NO: 24

GACATCCAGATGACCCAGAGCCCTAGCAGCCTGAGCGCTAGCGTGGGCGA
CAGGGTGACCATCACCTGCAGGGCCAGCCAGGGCATCAGAAACGACCTGG
GCTGGTACCAGCAGAAGCCCGGCAAGGCCCCCAAGCTGCTGATCTACGCC
GCCAGCAGCCTGCAGAGCGGAGTGCCTAGCAGGTTCAGCGGAAGCGGCAG
CGGCACCGACTTCACCCTGACCATCAGCAGCCTGCAGCCCGAGGACTTCG
CCACCTACTACTGCCTGCAGGACTACAACTACCCCCTGACATTCGGCGGC
GGCACCAAGGTGGAGATCAAGCGTACGGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGTTAG

### VH, VL, HC and LC of Ab1 CD3 Binding Domain

### VH Amino Acid Sequence of TMCB150 CD3 Binding Domain; SEQ ID NO: 17

QVQLQQSGPRLVRPSQTLSLTCAISGDSVFNNNAAWSWIRQSPSRGLEWL
GRTYYRSKWLYDYAVSVKSRITVNPDTSRNQFTLQLNSVTPEDTALYYCA
RGYSSSFDYWGQGTLVTVSS

### VL Amino Acid Sequence of TMCB150 CD3 Binding Domain; SEQ ID NO: 18

QSALTQPASVSGSPGQSITISCTGTSSNIGTYKFVSWYQQHPDKAPKVLL
YEVSKRPSGVSSRFSGSKSGNTASLTISGLQAEDQADYHCVSYAGSGTLL
FGGGTKLTVL

### HC Amino Acid Sequence of TMCB150 CD3 Binding Domain; SEQ ID NO: 19

QVQLQQSGPRLVRPSQTLSLTCAISGDSVENNNAAWSWIRQSPSRGLEWL
GRTYYRSKWLYDYAVSVKSRITVNPDTSRNQFTLQLNSVTPEDTALYYCA
RGYSSSFDYWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEAAGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFLLYSKLTVDKSRWQEGNVFSCSVMHEALHNHYTQKSLSLSLGK

### LC Amino Acid Sequence of TMCB150 CD3 Binding Domain; SEQ ID NO: 20

QSALTQPASVSGSPGQSITISCTGTSSNIGTYKFVSWYQQHPDKAPKVLL
YEVSKRPSGVSSRFSGSKSGNTASLTISGLQAEDQADYHCVSYAGSGTLL
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

### HC Nucleic Acid Sequence of TMCB150 CD3 Binding Domain; SEQ ID NO: 25

CAGGTGCAGCTGCAGCAGTCTGGCCCTAGACTCGTGCGGCCTTCCCAGAC
CCTGTCTCTGACCTGTGCCATCTCCGGCGACTCCGTGTTCAACAACAACG
CCGCCTGGTCCTGGATCCGGCAGAGCCCTTCTAGAGGCCTGGAATGGCTG
GGCCGGACCTACTACCGGTCCAAGTGGCTGTACGACTACGCCGTGTCCGT
GAAGTCCCGGATCACCGTGAACCCTGACACCTCCCGGAACCAGTTCACCC
TGCAGCTGAACTCCGTGACCCCTGAGGACACCGCCCTGTACTACTGCGCC
AGAGGCTACTCCTCCTCCTTCGACTATTGGGGCCAGGGCACCCTCGTGAC
CGTGTCCTCTGCTTCCACCAAGGGCCCATCCGTCTTCCCCCTGGCGCCCT
GCTCCAGGAGCACCTCCGAGAGCACAGCCGCCCTGGGCTGCCTGGTCAAG
GACTACTTCCCCGAACCGGTGACGGTGTCGTGGAACTCAGGCGCCCTGAC
CAGCGGCGTGCACACCTTCCCGGCTGTCCTACAGTCCTCAGGACTCTACT
CCCTCAGCAGCGTGGTGACCGTGCCCTCCAGCAGCTTGGGCACGAAAACC
TACACCTGCAACGTAGATCACAAGCCCAGCAACACCAAGGTGGACAAGAG
AGTTGAGTCCAAATATGGTCCCCCATGCCCACCATGCCCAGCACCTGAGG
CCGCCGGGGGACCATCAGTCTTCCTGTTCCCCCCAAAACCCAAGGACACT
CTCATGATCTCCCGGACCCCTGAGGTCACGTGCGTGGTGGTGGACGTGAG
CCAGGAAGACCCCGAGGTCCAGTTCAACTGGTACGTGGATGGCGTGGAGG
TGCATAATGCCAAGACAAAGCCGCGGGAGGAGCAGTTCAACAGCACGTAC
CGTGTGGTCAGCGTCCTCACCGTCCTGCACCAGGACTGGCTGAACGGCAA
GGAGTACAAGTGCAAGGTCTCCAACAAAGGCCTCCCGTCCTCCATCGAGA
AAACCATCTCCAAAGCCAAAGGGCAGCCCCGAGAGCCACAGGTGTACACC
CTGCCCCCATCCCAGGAGGAGATGACCAAGAACCAGGTCAGCCTGACCTG
CCTGGTCAAAGGCTTCTACCCCAGCGACATCGCCGTGGAGTGGGAGAGCA
ATGGGCAGCCGGAGAACAACTACAAGACCACGCCTCCCGTGCTGGACTCC
GACGGCTCCTTCCTCCTCTACAGCAAGCTAACCGTGGACAAGAGCAGGTG
GCAGGAGGGGAATGTCTTCTCATGCTCCGTGATGCATGAGGCTCTGCACA
ACCACTACACACAGAAGAGCCTCTCCCTGTCTCTGGGTAAA

### LC Nucleic Acid Sequence of TMCB150 CD3 Binding Domain; SEQ ID NO: 26

CAGTCTGCTCTGACCCAGCCTGCCTCCGTGTCTGGCTCTCCCGGCCAGTC
CATCACCATCAGCTGTACCGGCACCTCCTCCAACATCGGCACCTACAAGT
TCGTGTCCTGGTATCAGCAGCACCCCGACAAGGCCCCCAAAGTGCTGCTG
TACGAGGTGTCCAAGCGGCCCTCTGGCGTGTCCTCCAGATTCTCCGGCTC
CAAGTCTGGCAACACCGCCTCCCTGACCATCAGCGGACTGCAGGCTGAGG
ACCAGGCCGACTACCACTGTGTGTCCTACGCTGGCTCTGGCACCCTGCTG
TTTGGCGGAGGCACCAAGCTGACCGTGCTGGGTCAGCCCAAGGCTGCACC
CAGTGTCACTCTGTTCCCGCCCTCCTCTGAGGAGCTTCAAGCCAACAAGG
CCACACTGGTGTGTCTCATAAGTGACTTCTACCCGGGAGCCGTGACAGTG
GCCTGGAAGGCCGATAGCAGCCCCGTCAAGGCGGGAGTGGAGACCACCAC
ACCCTCCAAACAAAGCAACAACAAGTACGCGGCCAGCAGCTATCTGAGCC
TGACGCCTGAGCAGTGGAAGTCCCACAGAAGCTACAGCTGCCAGGTCACG
CATGAAGGGAGCACCGTGGAGAAGACAGTGGCCCCTACAGAATGTTCA

The term ‘study drug’ refers to Ab1 described in herein and refers to the study drug used in the Pharmacodynamic study described in Example 2 and the clinical trial protocol described in Example 3.

### Example 2. Preclinical Pharmacodynamic Study in Cynomolgus Monkeys

The pharmacodynamics of the study drug was evaluated in cynomolgus monkeys to understand the dosing and on-target effects of the study drug in prostate.

### Study Design

Two dosing regimens of intravenous study drug were evaluated in male cynomolgus monkeys (aged 3 to 8 years): single, fixed dosing (0.075 mg/kg) and step-up dosing (0.075 mg/kg, followed by 0.3 mg/kg 1 week later). Each dosing regimen included 12 male cynomolgus monkeys which were divided equally into 4 groups to receive study drug or control. Prostatic tissue, blood, and serum were collected for further analysis 24, 72 and 168 hours after administration of the fixed dose of 0.075 mg/kg and 24, 72 and 168 hours after administration of the 0.3 mg/kg dose of the step-up dosing regimen.

### Sample Collection

For study drug fixed dosing, whole blood was collected predose, 6 hours post-dose, and immediately proceeding necropsy at all further time points (24 hours, 72 hours, and 168 hours). Prostate tissues were collected at the three necropsy time points.

For study drug step-up dosing, samples were collected at timepoints similar to fixed dosing, with some slight adjustments. Whole blood was collected prior to priming dose and 6 hours thereafter, as well as prior to step-up dose one week later and 6 hours thereafter. Whole blood collections immediately proceeding necropsy were at 24 hours, 72 hours, and 168 hours post step-up dose. Prostate tissues were collected as described for fixed dosing.

All samples were processed by Flow Cytometry.

### Cell Surface Staining

Whole blood samples were collected, prepared, and stained for CD4, CD8, CD25, CD45, CD69, PD-1, Ki-67, FoxP3, and Granzyme B per standard procedures. Prostate tissues were minced into smaller pieces and processed as per standard procedures for further analysis. Data analysis was performed using FlowJo version 10 to determine frequency and counts. Cell enumeration was facilitated by addition of a constant volume of Precision Counting Beads. Conversion of acquired cellular events to cells/mL was calculated as follows:

$$Concentration of Beads [ 99700 \frac{beads}{ml} ] \times \frac{Count of Population \times Volume of Beads [ 5 ul ]}{Count of Beads \times Volume of Cell Suspension [ 195 ul ]}$$

### Concentrations of Study Drug

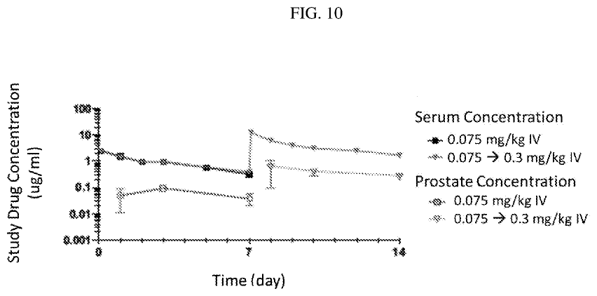

Validated and fit-for-purpose ECLIA (electrochemiluminescence immunoassay) method on the MSD® platform was used for quantifying the study drug concentrations in serum and prostate tissue, respectively. Lowest quantifiable concentration for serum and prostate tissue lysate samples were 0.01 μg/mL and 1.25 ng/ml, respectively. Approximately dose proportional increase of study drug was observed after step-up dosing (FIG. 10). Median peak study drug exposure in prostate was 11.0% of serum for first dose and 8.7% after second dose.

### T Cell Infiltration

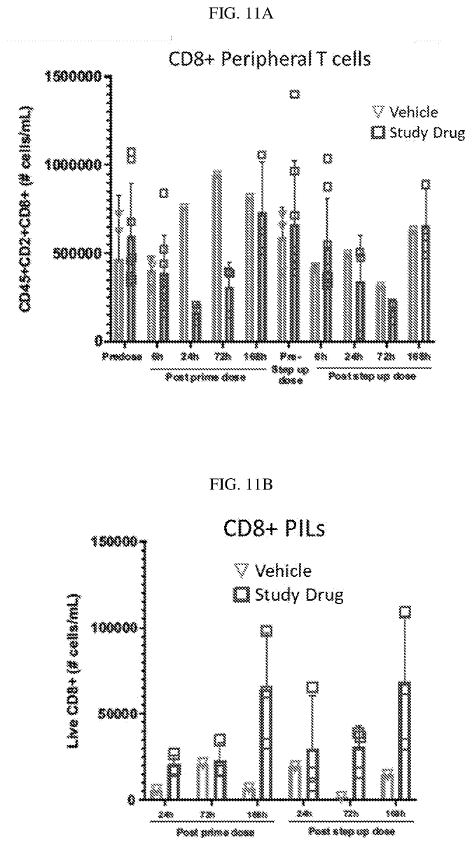

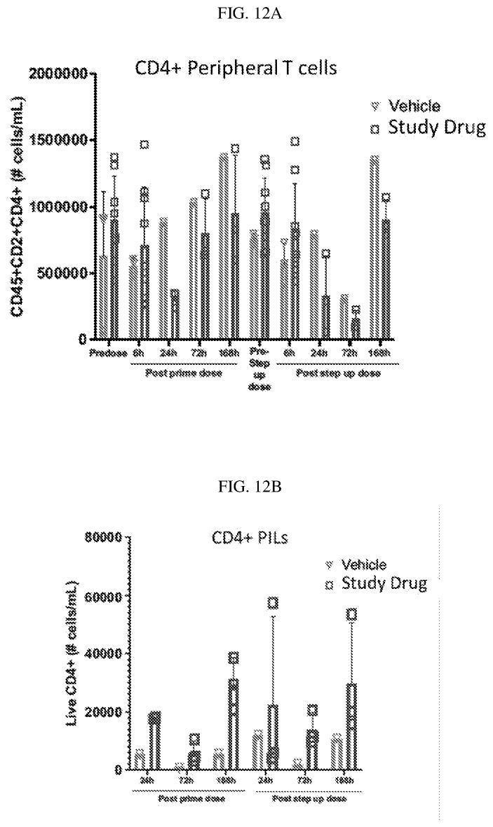

Study drug dosing increased prostate infiltration of CD8+ T cells and CD4+ T cells and reduced peripheral T cells (FIG. 11A-11B, FIG. 12A-12F). Reduction of peripheral CD8+ T cells was more pronounced with study drug fixed dosing.

### T Cell Activation and Proliferation

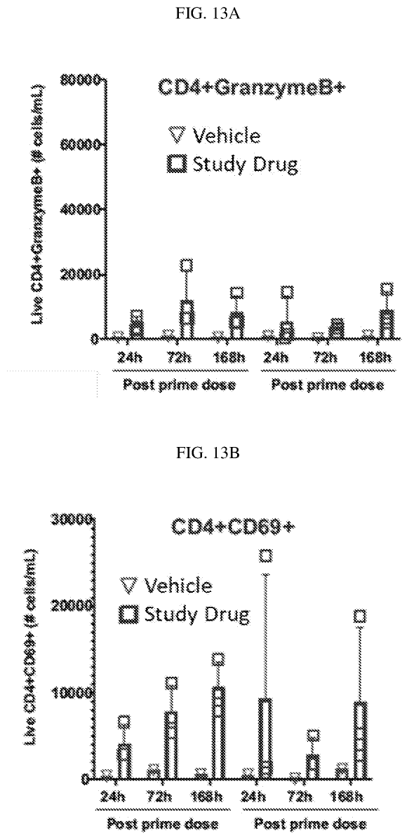

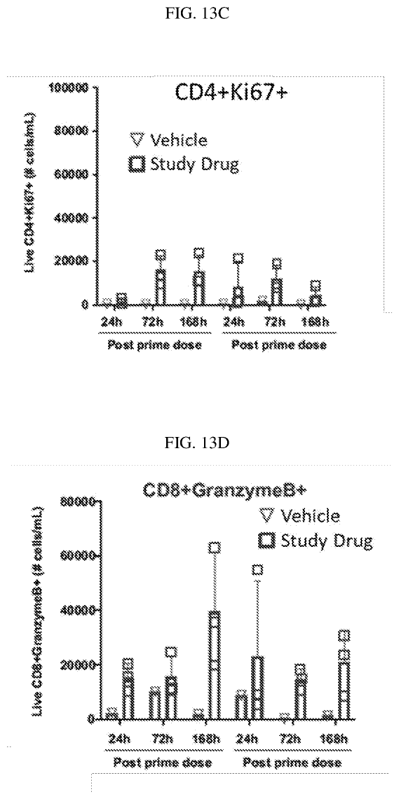

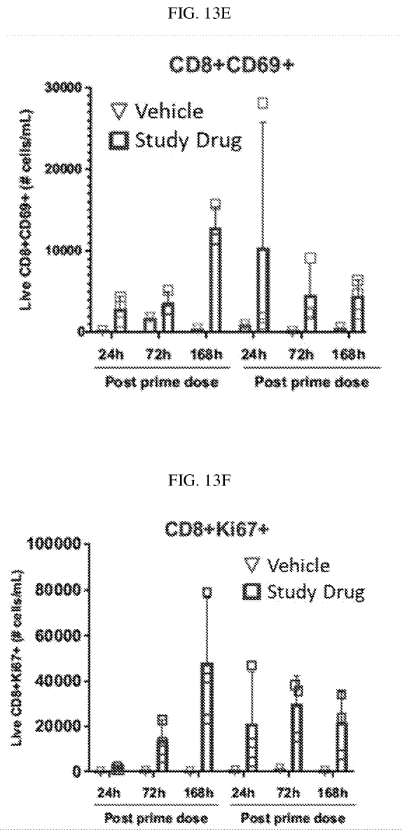

Both CD4+ and CD8+ prostatic T cells expressed markers of activation and proliferation upon study drug dosing (FIG. 13A-13F). The magnitude of T cell activation seemed slightly attenuated with the Study Drug step-up dosing.

### Suppressive T Cell Markers

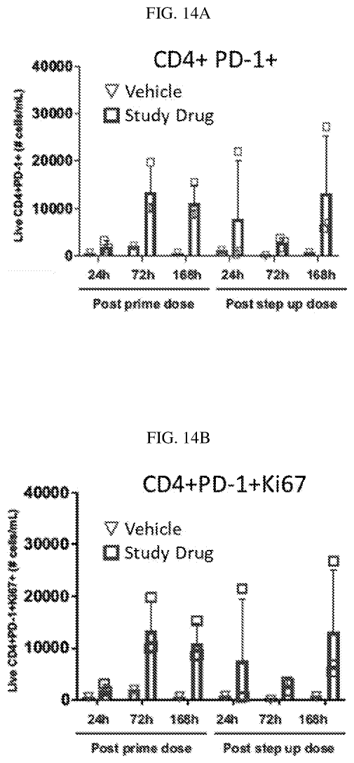

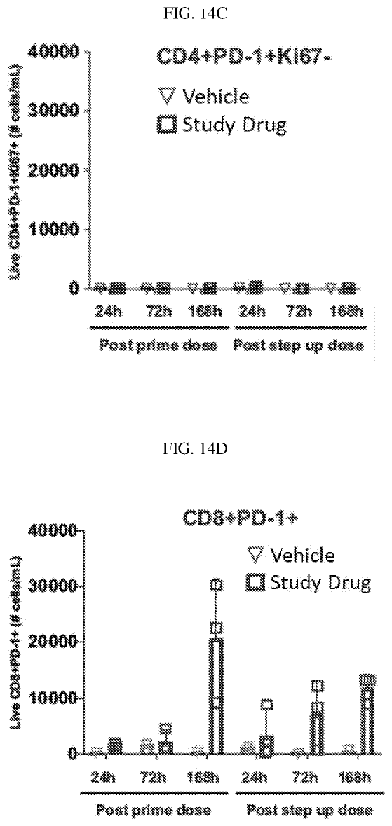

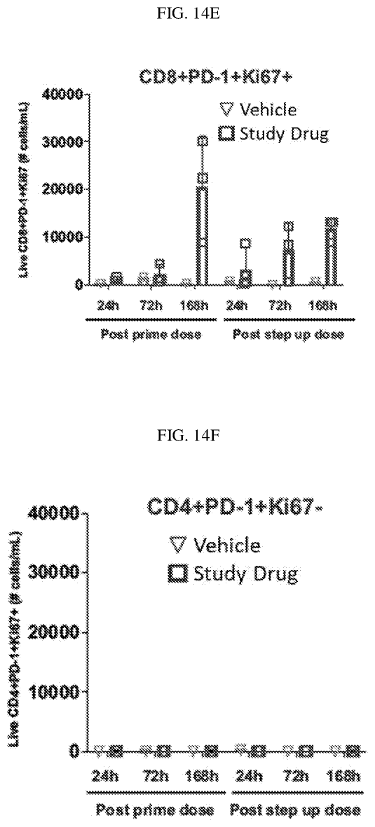

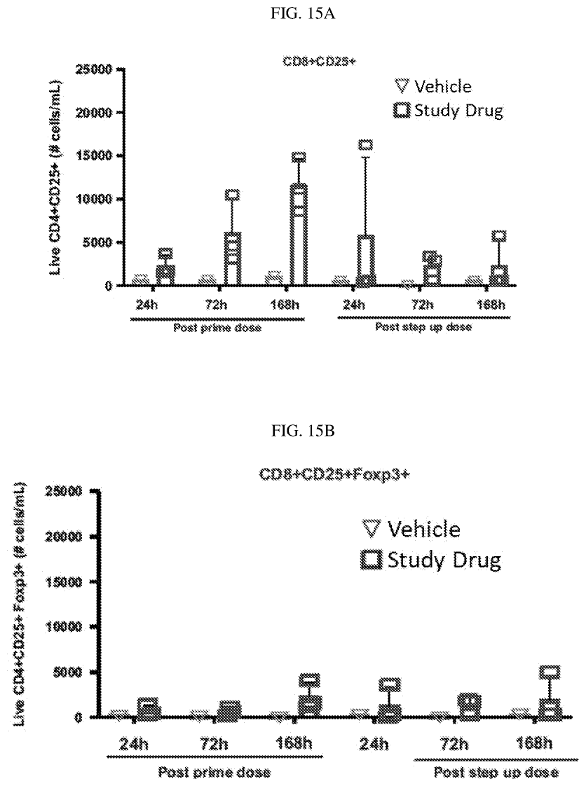

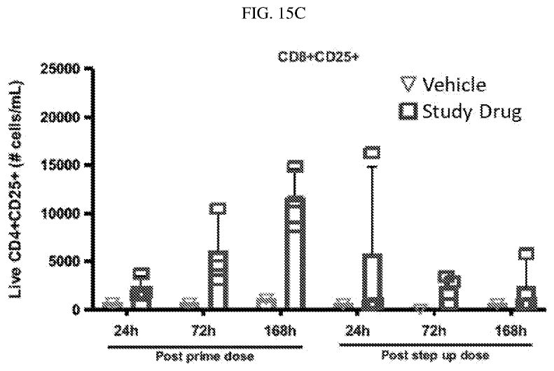

The study drug had minimal effects on suppressive T cell markers (PD-1+Ki67−, CD4+CD25+Foxp3) (FIG. 14A-14C and FIG. 15A-15B)

### Myeloid Cell Infiltration

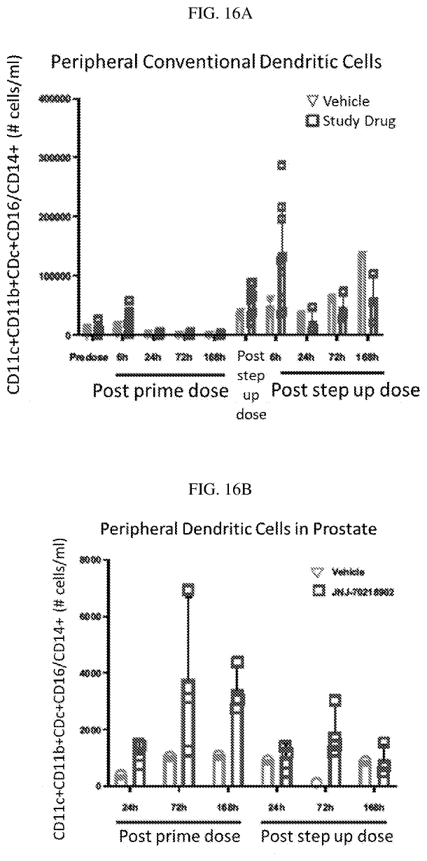

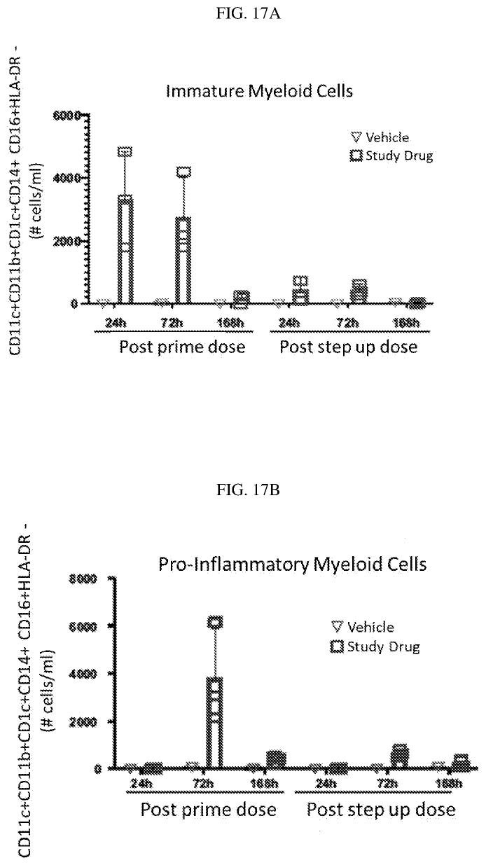

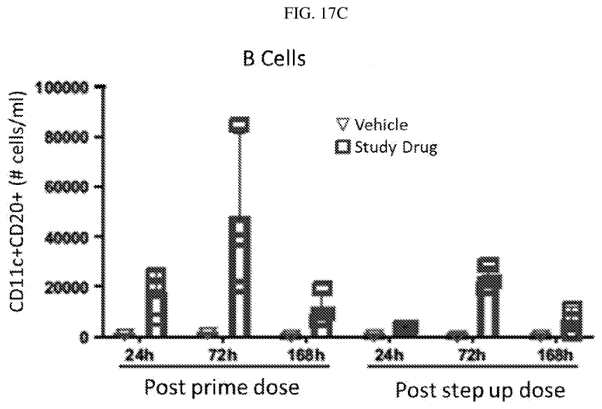

Study drug dosing led to influx of pro-inflammatory cells (dendritic cells, myeloid cells, B cells) (FIG. 16A-16B, FIG. 17A-17C). Study drug step-up dosing showed decreased immature and pro-inflammatory myeloid subpopulations in prostate tissues compared with fixed dosing. Total B cell numbers were lower with study drug step-up dosing compared with fixed dosing.

### Conclusions

Study drug dosing increased intra-prostatic T cell numbers. T cell activation and proliferation were observed in a target-specific manner, and no significant activation or proliferation of T cells were detected in the periphery. Suppressive T cell markers (PD1+Ki-67− and CD25+FoxP3) were not seen in significant numbers after study drug dosing. Myeloid cell infiltrates support an active immune microenvironment in prostate following study drug dosing.

### Example 3. Phase 1 Clinical Study

### Protocol Summary

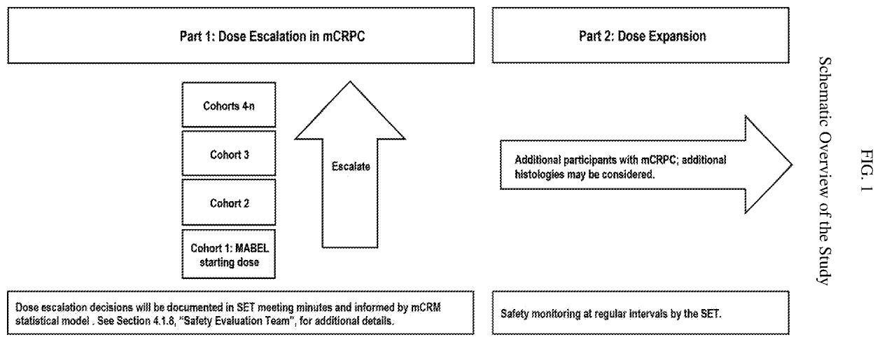

This is a first-in-human (FIH), open-label, multicenter, Phase 1 dose escalation study to evaluate the safety, pharmacokinetics (PK), pharmacodynamics (PD), and preliminary clinical activity of study drug monotherapy in participants with metastatic castration-resistant prostate cancer (mCRPC). A diagram of the study design is provided in FIG. 1.

Dose escalation was supported by a modified continual reassessment method (mCRM). The goal was to determine the maximum tolerated dose (MTD) of the study drug.

During the study, safety was monitored at regular intervals by the Study Evaluation Team (SET), particularly at each dose escalation step. The study was initiated with a once-weekly (QW) dosing schedule via subcutaneous (SC) injection. An alternative dosing schedule of once every 2 weeks [Q2W] was explored based on emerging data as determined by the SET.

Approximately 73 participants were treated in this study.

TABLE 22

| | |
|---|---|
| Starting dose/ | Dose escalation was initiated at a starting |
| Dose levels | dose of 300 μg administered SC. |
| Route of | The route of administration is SC injection. |
| administration | |
| Dosing | The study was initiated with a once-weekly |
| Schedule/ | dosing schedule. The study drug administration |
| Regimen | schedule may be changed to once every 2 weeks. |

### Objectives, Endpoints

The study objectives and endpoints of the study are listed in Table 33.

TABLE 33
Objectives and endpoints of the study

| | |
|---|---|
| Objectives | Endpoints |

Primary

| | |
|---|---|
| Dose Escalation | |
| Determine the recommended | Incidence and severity of all adverse |
| Phase 2 dose(s) (RP2D[s])/ | events (AEs), including incidence of |
| regimen(s) and the maximum | dose-limiting toxicity (DLTs); |
| tolerated dose (MTD; if | |
| possible). | |
| Secondary | |
| To assess the PK, PD, | PK and PD parameters, anti- study drug |
| immunogenicity and | antibodies and preliminary response |
| preliminary clinical activity | based on Prostate Cancer Working |
| following multiple doses. | Group 3 (PCWG3) criteria or RECIST |
| | 1.1. |

### Efficacy Evaluation

Clinical activity will be assessed using the following evaluations: computed tomography (CT) scan of the chest, abdomen, and pelvis with contrast as clinically indicated; magnetic resonance imaging (MRI) may be substituted as clinically indicated (ie, for sites not adequately imaged using CT). Additional evaluations for participants with mCRPC include serum prostate-specific antigen (PSA) and whole-body bone scans (^{99m}Tc). Evaluation of treatment response will be performed according to Prostate Cancer Working Group 3 (PCWG3) criteria and Response Evaluation Criteria in Solid Tumors (RECIST) version 1.1 to evaluate progression of soft tissue lesions (CT or MRI).

### Pharmacokinetic, Biomarker, and Immunogenicity Evaluations

Blood samples will be collected to characterize serum pharmacokinetics and anti-drug antibodies of the study drug. Blood samples will also be collected to evaluate pharmacodynamics, safety, and biomarkers predictive of response or resistance to the study drug treatment. To evaluate TMEFF2 expression as well as T-cell infiltration, archival tissue samples from metastatic (non-prostate) tumor lesions (collected at any time prior to enrollment) as well as archival prostate samples collected ≤15 months from providing informed consent will be requested if available. In addition, selected participants in PK/PD cohorts in Part 1 and all participants in Part 2 will undergo fresh tumor biopsies to evaluate pharmacodynamic markers in tumor tissue. Selected PK/PD cohorts may undergo additional blood sample collections.

### Safety Evaluation

The safety of the Drug Study will be assessed by physical examinations (including neurological), vital signs, Eastern Cooperative Oncology Group (ECOG) performance status, clinical laboratory tests, electrocardiograms (ECGs), ophthalmological examinations and adverse event (AE) monitoring (including dose-limiting toxicities [DLTs], serious adverse events [SAEs] and adverse events of special interest [AESI]). Concomitant medication usage will be recorded. The severity of adverse events will be assessed using National Cancer Institute Common Terminology Criteria for Adverse Events (Version 5.0) except for cytokine release syndrome (CRS), which will be graded according to the American Society for Transplantation and Cellular Therapy (ASTCT) guidelines. Central and peripheral neurotoxicity (Grade ≥3), and new onset retinal abnormalities or noninfectious inflammatory ocular disorders (Grade ≥2) have been identified as adverse events of special interest and will require enhanced reporting and data collection.

### Starting Dose Rationale

The first-in-human (FIH) starting dose of 300 μg via SC administration approximates a 4 μg/kg dose (with assumed median body weight of 75 kg and 100% bioavailability).

In vitro cytotoxicity assays were conducted to characterize the study drug-induced T cell activation, TMEFF2-positive tumor cell killing, and cytokine release. These assays were conducted with purified human T cells from healthy human donors and LNCaP-AR, a human prostate cancer cell line that expresses TMEFF2 and demonstrates sensitivity to T cell mediated killing. Median cytotoxicity was shown to be the most sensitive and was considered the most predictive for T cell mediated killing (Table 4). The MABEL concentration of 1.0 nM (0.15 μg/mL) was determined from the median EC₂₀ value of cytotoxicity.

TABLE 4
Summary of Exposure-response Analysis of T
Cell-mediated Cytotoxicity and T Cell Activation
Assays with study drug using LNCaP-AR Cells

| | | |
|---|---|---|
| N (Number of Donors) | Median (nM) | Range (nM) |

T cell Activation

| | | |
|---|---|---|
| 6 | 2.0954 | 1.2329-2.6853 |

Cytotoxicity

| | | |
|---|---|---|
| 5 | 1.0301 | 0.7083-1.553 |

EC₂₀ (nM) = drug concentration required to produce 20% of the maximal effect

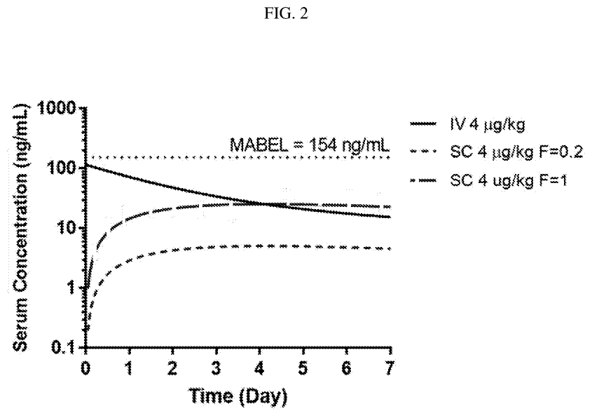

PK Model MABEL predicted mean study drug serum concentration in human following a single Administration at 300 μg Assuming Median Body Weight of 75 kg is shown in FIG. 2.

Based on overall assessment of in vitro and in vivo data and MABEL-based FIH starting dose selection, 300 μg weekly SC dose of the study drug should result in drug exposure that has minimal biological activity in participants treated in this study.

### Study Design

### Overall Design

This is a FIH, open-label, multicenter, Phase 1 study to evaluate the safety, pharmacokinetics, pharmacodynamics, and preliminary clinical activity of study drug monotherapy in participants with mCRPC. Approximately 73 participants were treated in this study. Once a participant is determined to be eligible (ie, inclusion/exclusion criteria) for the study and has provided informed consent for study participation, study drug will be administered as a SC injection.

The pharmacodynamics of the study drug were further characterized in selected PK/PD cohorts as determined by the Study Evaluation Team (SET).

A diagram of the study design is provided in FIG. 1.

### Dose Escalation

The study was designed to select the RP2D(s) and regimen(s) and to determine the MTD (if possible) of the study drug in participants with mCRPC. Dose escalation began at the MABEL-based starting dose of 300 μg (0.3 mg) and proceed as shown in FIG. 1. Only the starting dose was pre-specified. After the starting dose, subsequent doses were selected based on the review of all available data including, but not limited to, pharmacokinetic, pharmacodynamic, safety, and preliminary clinical activity. Dose escalation decision-making was guided by the modified continual reassessment method (mCRM).

Multiple dose levels were enrolled in parallel with each new dose level/schedule recommended by the SET and supported by the statistical model with Escalation with Overdose Control (EWOC) principle. To better understand the safety, tolerability, pharmacokinetics, pharmacodynamics, or preliminary antitumor activity, additional participants were enrolled at one or more dose cohorts (referred to as PK/PD cohorts) at or below doses considered to be safe by the SET.

Dose escalation was carried out in sequential cohorts of single or multiple participants at doses assigned by the SET. The following guidelines applied during dose escalation:

If more than 1 participant was treated at a dose level, the first participant treated at that given dose level were observed for a minimum of 2 days prior to treating subsequent participants.

Escalation proceeded with doses increasing by not more than half-log increments.

Dose escalation decisions were made by the SET based on a mCRM utilizing all the available data, such as safety, pharmacodynamic, pharmacokinetic, and other biomarker(s) data of all prior dose levels. Preliminary clinical activity, if available, was reviewed by the SET during dose escalation.

### Part 2 (Dose Expansion)

In Part 2, the RP2D(s)/regimen(s) of the study drug as determined in Part 1 will be administered to additional participants with mCRPC to confirm the safety, pharmacokinetics, pharmacodynamics, and preliminary clinical activity of the study drug. Additional histologies (in addition to mCRPC) will be considered for dose expansion based on emerging data from Part 1.

### Treatment Dose Schedule

The study was initiated with Q1W treatment doses. An Alternative dosing schedule of Q2W was evaluated based on emerging safety and pharmacokinetic data after approval by the SET.

Prior to the first dose of study drug, corticosteroid, antihistamine, and antipyretic premedications was administered to minimize the risks associated with cytokine release syndrome (CRS) and infusion-related reactions. The premedication dose(s) or schedule(s) was reduced or omitted for subsequent doses based on SET review of available data; on a case-by-case basis, corticosteroid premedication was reduced or omitted for subsequent doses. For participants who experience a Grade 2 or higher CRS or IRR, pretreatment corticosteroid was required for at least 1 subsequent dose administered to that participant.

### Determination of Recommended Phase 2 Dose (RP2D)

The RP2D(s) will be determined after review of all available data including safety, pharmacokinetic, pharmacodynamic and clinical activity from at least 6 participants treated at the dose levels being considered for RP2D declaration. Additionally, pharmacokinetic data from at least 12 participants across all dose levels, as well as the recommended dose by Bayesian Logistic Regression Model (BLRM), will be taken into consideration.

### Definition of Dose Limiting Toxicity (DLT)

The DLT evaluation period is defined as the first 21 days of treatment.

TABLE 5
Dose-Limiting Toxicity Criteria
Hematologic Toxicity

| | |
|---|---|
| Neutrophil count | Febrile neutropenia |
| decreased | Neutropenia: Grade 4 for ≥7 days |
| Platelet count | Grade ≥3 thrombocytopenia with bleeding or any |
| decreased | Grade 4 thrombocytopenia with duration ≥7 days |
| Any hematological | Grade 5 |
| toxicity | |

Non-hematological Toxicity
Any non-hematological toxicity of Grade ≥3, with the following exceptions:
Grade 3 fatigue, fever, constipation, nausea, vomiting, or diarrhea lasting
<5 days with best supportive care;
Grade ≥3 hypertension that can be controlled by medical management;
Grade ≥3 ALT or AST that resolves to Grade ≤1 or baseline within 7 days,
unless criteria for Hy's law are met^{c};
Isolated Grade ≥3 alkaline phosphatase or GGT increase that returns to
Grade ≤1 or baseline within 7 days;
Grade ≥3 lipase or amylase increase not associated with clinical or
radiological evidence of pancreatitis;
Grade ≥3 electrolyte abnormalities^{d} that resolve spontaneously within
7 days, or that respond to best supportive care;
First occurrence of Grade ≥3 IRR or CRS.
Abbreviations:
ALP = alkaline phosphatase;
ALT = alanine aminotransferase;
AST = aspartateaminotransferase;
DLT = dose-limiting toxicity;
GGT = gamma-glutamyl transferase;
IRR = infusion-related reactions;
ULN = upper limit of normal

### Route of Administration

The study explored SC routes of administration.

### Treatment Discontinuation/Follow-Up

Participants received study drug until radiographic disease progression, unequivocal clinical progression, unacceptable toxicity, or any other treatment discontinuation criteria were met. After treatment discontinuation, participants clinically able to return for evaluation has an End-of-Treatment (EOT) Visit within 30 (+7) days after the last dose of study drug.

### Study Population

The inclusion and exclusion criteria for enrolling participants in this study are described below.

### Inclusion Criteria

Each potential participant satisfied all the following criteria to be enrolled in the study:
≥18 years of age;
Histology: Metastatic CRPC (mCRPC) with histologic confirmation of adenocarcinoma. Adenocarcinoma with small-cell or neuroendocrine features is allowed;
Measurable or evaluable disease;
Prior treatment with at least 1 prior novel AR-targeted therapy (ie, abiraterone acetate, apalutamide, enzalutamide, darolutamide), or chemotherapy (eg, docetaxel);
Eastern Cooperative Oncology Group (ECO) performance status grade of 0 or 1;
Hematology laboratory parameters within the following ranges, independent of transfusion within 7 days or growth factors within 3 weeks prior to first dose of study drug. Participant must not be transfusion-dependent (Hemoglobin ≥9 g/dL, Absolute neutrophil count ≥1.5×10⁹/L, Platelets count ≥75×10⁹/L);
Chemistry laboratory parameters within the following range (Serum albumin ≥3.0 g/dL, Calculated or measured creatinine clearance >50 mL/min/1.73 m², Serum total bilirubin ≤1.5×the upper limit of normal (ULN); in participants with Gilbert's syndrome, if total bilirubin is ≥1.5×ULN, measure direct and indirect bilirubin and if direct bilirubin is within the normal limit, participant may be eligible, Aspartate aminotransferase (AST) and alanine aminotransferase (ALT)≤2.5×ULN (or ≤4×ULN for participants with tumor involvement in the liver)

### Exclusion Criteria

Any potential participant who met any of the following criteria were excluded from participating in the study:
Known brain metastases.
Concurrent use of any other anticancer treatment (including non-palliative radiotherapy) or investigational agent; prior anticancer treatments (including non-palliative radiotherapy) must be discontinued for at least 2 weeks before the first dose of study drug.
Toxicities related to prior anticancer treatments have not returned to Grade ≤1 or baseline, except for alopecia and vitiligo.
Symptomatic diabetic retinopathy with macular edema or active wet, age related macular degeneration (AMD), and any symptomatic eye inflammation or optic nerve disorders (eg, uveitis, optic neuritis) within previous 12 months.
Solid organ or bone marrow transplantation.
Seizures or known condition that may predispose to seizures or intracranial masses such as schwannomas and meningiomas that are causing edema or mass effect.
Any of the following within 6 months prior to signature of informed consent:
a. Myocardial infarction
b. Severe or unstable angina
c. Clinically significant ventricular arrhythmias
d. Congestive heart failure (New York Heart Association class III to IV)
e. Transient ischemic attack
f. Cerebrovascular accident.
Venous thromboembolic events (ie, pulmonary embolism) within 1 month prior to the first dose of study drug; uncomplicated (Grade ≤2) deep vein thrombosis is not considered exclusionary.
Peripheral neuropathy or neuropathic pain of Grade ≥2.
Clinically significant pulmonary compromise, particularly a requirement for supplemental oxygen use (>2 L by nasal cannula) to maintain adequate oxygenation.
Known allergies, hypersensitivity, or intolerance to the study drug or its excipients
Concurrent use of any other anticancer treatment or investigational agent for the treatment of advanced disease.
Active infection or condition that requires treatment with systemic anti infective agents within 7 days prior to the first dose of study drug.
Received immunosuppressive doses of systemic medications, such as corticosteroids (doses >10 mg/day prednisone or equivalent) within 3 days prior to the first dose of study drug. A single course of corticosteroids is permitted as prophylaxis for imaging contrast (ie, for participants with allergies to contrast). If corticosteroids were used to treat immune-related adverse events associated with prior therapy, ≥7 days must have elapsed since the last dose of corticosteroid.
Active autoimmune disease within the 12 months prior to signing consent that requires systemic immunosuppressive medications (eg, chronic corticosteroid, methotrexate, or tacrolimus).
Had major surgery (eg, requiring general anesthesia). Participant must have recovered adequately without sequelae at least 3 weeks prior to the first dose of study drug.
Active or chronic hepatitis B or hepatitis C infection. Hepatitis B infection defined by a positive test for hepatitis B surface antigen (HBsAg). Hepatitis C infection defined by a positive hepatitis C antibody.
Known history of Human Immunodeficiency Virus (HIV) antibody positivity.
Plans to father a child while enrolled in this study or within 90 days after the last dose of study drug.
Any condition for which, in the opinion of the investigator, participation would not be in the best interest of the participant (eg, compromise the well-being) or that could prevent, limit, or confound the protocol-specified assessments.
Received live or live attenuated vaccine(s) within 4 weeks prior to the first dose of study drug, during the study, or within 4 weeks after the last dose of study drug. Vaccines approved or authorized for emergency use (eg, COVID-19) and non-live vaccines (eg, influenza) are allowed.

### Study Drug Administration

The study was initiated with SC injection as route of administration, administered on a once-weekly dosing schedule. The alternative study drug administration schedule of Q2W was also explored. Dose escalation was initiated at a starting dose of 300 μg administered SC.

### Study Assessment and Procedure

### Efficacy Evaluation

Clinical activity was assessed using the following evaluations:

Identical methodology (CT scan or MRI and ^{99m}Tc bone scan) was used for disease assessment at baseline, and throughout the course of the study, to characterize each identified and reported lesion to document disease status. Ultrasound, [¹⁸F]-fluorodeoxyglucose positron emission tomography (PET), and plain X-rays were not acceptable methods of evaluating disease response. Imaging was not to be delayed due to delays in study drug administration. Efficacy evaluations include the following: PSA, whole-body bone scans (^{99m}Tc), CT scan or MRI. Evaluation of treatment response for prostate cancer was performed according to Prostate Cancer Working Group 3 (PCWG3) criteria.

Participants with an objective response per RECIST v1.1 must have had a confirmatory scan performed 4 weeks later. If a participant was assessed with partial response (PR) or complete response (CR) anytime during study drug treatment but without confirmation ≥4 weeks later, the participant's best response was classified as stable disease/progressive disease/not evaluable depending on the participant's next immediate assessments. During the study, disease response will be assessed using CT or MRI scans of the locations of known lesions.

### Assessment of Disease Response and Progressive Disease

### Soft Tissue Lesion Assessment (CT or MRI, Physical Examination)

Baseline disease burden was assessed using CT scans of the chest, abdomen, and pelvis, plus other areas as appropriate, with IV contrast. Participants who were intolerant of IV contrast agents may have had CT scans performed with oral contrast and the reason for not using IV contrast was documented in source documents. Subsequent efficacy evaluations during the study included radiographic imaging of all disease sites documented at baseline.

Magnetic resonance imaging was used to evaluate sites of disease that could not be adequately imaged using CT (in any case where an MRI is desirable, it must be the imaging technique used to assess disease at baseline and at all subsequent response evaluations). For all other sites of disease, MRI assessments did not replace the required chest, abdomen, and pelvic CT scans, unless CT scan was contraindicated. Brain MRI was required only if clinically indicated. CT scan of the head was used if MRI was contraindicated.

For participants with palpable/superficial lesions, clinical disease assessments by physical examination were performed at baseline and throughout study drug treatment, as clinically indicated. Irradiated or excised lesions were considered not measurable and monitored only for disease progression.

### Bone Lesion Assessment in Prostate Cancer

Bone disease for participants with prostate cancer was evaluated according to PCWG3 (ie, to evaluate duration of response) as follows:
Progression of soft tissue lesions measured by CT or MRI as defined in RECIST v1.1.
Progression by bone lesions observed by bone scan and based on PCWG3. Under these criteria, any bone progression must be confirmed by a subsequent scan ≥6 weeks later. The Week 8 scan (first post-treatment scan) should be used as the reference scan to which all subsequent scans are compared to determine progression. Bone progression is defined as one of the following:
   1. Participant whose Week 8 scan is observed to have ≥2 new bone lesions compared to baseline scan will need to have a confirmatory scan performed ≥6 weeks later and would fall into one of the 2 categories below:
      a. Participant whose confirmatory scan (which is performed ≥6 weeks later) shows ≥2 new lesions compared to the Week 8 scan (ie, a total of ≥4 new lesions compared to baseline scan) will be considered to have bone scan progression at Week 8.
      b. Participant whose confirmatory scan did not show ≥2 new lesions compared to the Week 8 scan will not be considered to have bone scan progression at that time. The Week 8 scan will be considered as the reference scan to which subsequent scans are compared.
   2. For a participant whose Week 8 scan does not have ≥2 new bone lesions compared to baseline scan, the first scan timepoint that shows ≥2 new lesions compared with the Week 8 scan will be considered as the bone scan progression timepoint if these new lesions are confirmed by a subsequent scan ≥6 weeks later.

### Pharmacokinetic and Immunogenicity Evaluations

Venous blood samples were collected for measurement of serum concentrations of the study drug and anti-study drug antibodies. Blood samples were obtained from the arm contralateral to the arm where the study drug was infused when study drug was administered via peripheral vein. At timepoints where both serum concentration and immunogenicity was evaluated, 1 blood draw was collected and the serum sample was divided into separate aliquots. Samples collected for analyses of study drug serum concentration and antibody to the study drug may have additionally been used to evaluate safety or efficacy aspects that address concerns arising during or after the study period, for further characterization of immunogenicity or for the evaluation of relevant biomarkers (eg, possible presence of soluble TMEFF2).

### Population for Analyses

For purposes of analysis, the following populations are defined:
All Treated Analysis Set: This set consists of participants who received at least 1 dose of study drug. This analysis set will be considered as primary and will be used in all safety and efficacy summaries.
DLT Evaluable Analysis Set: This set is a subset of the ‘All Treated Analysis’ set. Participants who receive at least 75% of the planned doses of study drug during the DLT observation period as defined will be included in this analysis.
Biomarker Analysis Set: This set consists of all participants who received at least 1 dose of study drug and have at least 1 pre- or post-treatment biomarker measurement.
Pharmacokinetic Analysis Set: This set consists of all participants who receive at least 1 dose of study drug and have at least 1 evaluable concentration measurement of study drug.

### Statistical Analysis

### Efficacy Analyses

### Endpoint Definitions

PSA response rate (RR), defined as the proportion of participants with a decline of PSA of at least 30% or more from baseline. The maximal change at any time during the study was reported for each participant using waterfall plots. To reflect a change in clinical status, whenever possible, participants should remain in the study until radiographic or symptomatic progression. The analysis of the primary endpoint was performed on the all-treated population, and PSA RR was presented along with a 90% 2-sided exact CI.

Overall response rate (ORR) is defined as the proportion of participants who have a PR or better according to the RECIST v1.1 response criteria without evidence of bone progression according to PCWG3. Response to treatment was evaluated by investigator.

Duration of response (DOR) was calculated from the date of initial documentation of a response (PR or better) to the date of first documented evidence of progressive disease, as defined in the PCWG3 or RECIST v1.1 response criteria, or death due to any cause, whichever occurs first. For participants with a response (CR or PR) to treatment with disease that did not progress and who were alive, data was censored at the last disease evaluation before the start of any subsequent anticancer therapy.

Time to response (TTR) defined as the time from the date of first dose of study drug to the date of first documented response.

### Analysis Methods

Overall response rate was tabulated together with its two-sided 90% exact confidence interval. In addition, the number and percentage of participants in each response category was tabulated. For TTR, descriptive statistics were used to summarize the results, including mean, median, standard deviation, and range for participants with a response. For DOR, the Kaplan Meier-method was used for descriptive summaries.

### Safety Analyses

All safety analyses were performed on data from the ‘all treated analysis set’. The baseline value for safety assessment is defined as the value collected at the time closest to, but prior to, the start of the first study drug administration. The safety parameters that were evaluated are the incidence, severity, and type of adverse events, clinically significant changes in the participant's physical examination findings, vital signs measurements, clinical laboratory and other clinical test results (eg, ECG). Exposure to the study drug and reasons for discontinuation of study drug were tabulated. Adverse events were summarized by system organ class, preferred term, worst grade experienced by the participant, and by dose level. Safety was summarized by dose, route and schedule as appropriate.

### Adverse Events

The verbatim terms used in the CRF by investigators to identify adverse events were coded using the Medical Dictionary for Regulatory Activities (MedDRA). Study drug-emergent adverse events are adverse events with onset during the study drug phase or that are a consequence of a pre-existing condition that has worsened since baseline. All reported treatment-emergent adverse events were included in the analysis. For each treatment-emergent adverse event, the percentage of participants who experience at least 1 occurrence of the given event were summarized by dose level/dose cohort.

Summaries, listings, datasets, or participant narratives were provided, as appropriate, for those participants who die, who discontinue study drug due to an adverse event, or who experience a severe or a serious adverse event. Listings of DLTs used the DLT evaluable analysis set. DLTs were listed and the incidence summarized by primary system organ class, preferred term, worst grade and type of adverse event, and dose levels.

### Example 4. Safety and Preliminary Clinical Activity of the Study Drug for the Treatment of Metastatic Castrate-Resistant Prostate Cancer (mCRPC)

Study drug was administered subcutaneously (SC) at doses ranging from 0.3 mg to 6 mg once weekly (Q1W) and 2 to 6 mg once every 2 weeks (Q2W). A total of 9 dose levels were tested (Q1W: 300 ug, 1 mg, 1.5 mg, 3 mg, and 6 mg; Q2W: 2 mg, 3 mg, 4 mg, and 6 mg) with 82 total patients dosed.

### Pharmacokinetics

### Preliminary Pharmacokinetics

Preliminary PK of the study drug has been evaluated in 71 subjects from the ongoing FIH Study. Preliminary data after SC injection(s) of the study drug are available at doses ranging from 0.3 to 6.0 mg Q1W (Cohorts 1 to 4 and 9) and at doses ranging from 2.0 to 6.0 mg every 2 weeks (Q2W; Cohorts 5 to 8). Baseline characteristics are shown in Table 6.

TABLE 6
Baseline characteristics

| | Total |
|---|---|
| Age (years) | |
| N | 73 |
| Mean (SD) | 67 |
| Median (range) | 66 |
| Race | |
| N (%) | 73 |
| Asian | 2 (3) |
| White | 69 (95) |
| Multiple | 1 (1) |
| Unknown | 1 (1) |
| ECOG | |
| (%) | 73 |
| 0 | 31 (43) |
| 1 | 42 (58) |
| PSA at baseline (ug/L) | |
| N | 72^{c} |
| Mean (SD) | 238 (404) |
| Median (range) | 68 (0.21 15) |
| Extent of Disease | |
| N(%) | 73 |
| Bone | 65 (89) |
| Soft tissue | 54 (74) |
| Visceralª | 32 (44) |
| Lymph node^{b} | 39 (53) |
| Other | 10 (14) |
| Number of AR-targeted, | |
| taxane, cytotoxic chemotherapy, | |
| and other therapy | |
| N (%) | 68 |
| 1 | 3 (4) |
| 2 | 13 (19) |
| 3 | 17 (25) |
| 4 | 13 (19) |
| 5 | 15 (22) |
| 6 | 6 (9) |
| 10 | 1 (2) |

Abbreviations:
AR, antigen receptor,
ECOG, Eastern Cooperative Oncology Group:
PSA, prostate specific antigen;
SD, standard deviation.
ªincludes lung, liver, adrenal, and central nervous system, ^{b}includes pelvic and extra-pelvic, ^{c}PSA was not entered in database for 1 patient at time of data cut.

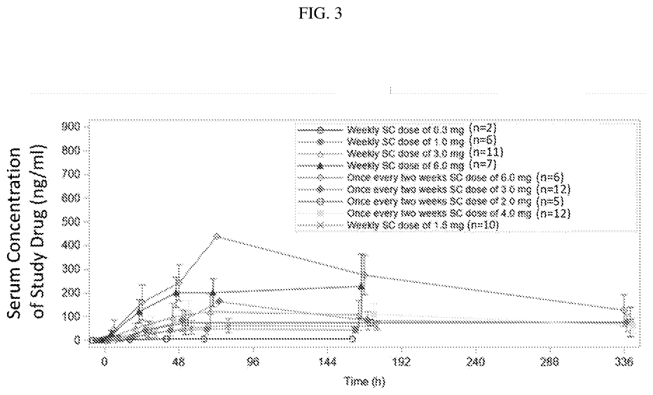

Following the first SC injection of the study drug ranging from 0.3 to 6.0 mg, both Cₘₐₓ and AUC₀₋₁₆₈ₕ increased in an approximately dose-proportional manner. A slow increase in the mean serum concentration of the study drug was observed for all dose levels (FIG. 3). The median Tₘₐₓ generally occurred between 72 and 168 hours (Table 7).

TABLE 7
Summary of Pharmacokinetic Parameters of study drug Following the First SC Injection

Pharmacokinetics

of

| study drug | | | | | | | | | |
|---|---|---|---|---|---|---|---|---|---|
| (mean [SD], Tₘₐₓ: | Weekly (Q1W) | | | | | Once every two weeks (Q2W) | | | |
| median [range]) | 0.3 mg | 1.0 mg | 1.5 mg | 3.0 mg | 6.0 mg | 2.0 mg | 3.0 mg | 4.0 mg | 6.0 mg |
| N | 2 | 6 | 8 | 10 | 7 | 5^{a} | 10^{b} | 11^{c} | 5^{d} |
| Cₘₐₓ, ng/mL | 7.25 | 51.7 | 66.1 | 134 | 249 | 98.0 | 95.1 | 123 | 315 |
| | | (20.6) | (29.0) | (77.3) | (130) | (51.4) | (47.7) | (51.2) | (116) |
| Tₘₐₓ, h | 120.00 | 120.0 | 72.00 | 72.00 | 72.00 | 168.00 | 168.00 | 168.00 | 168.00 |
| | (72.00- | (72.00- | (48.00- | (48.00- | (72.00- | (48.00- | (48.00- | (48.00- | (72.00- |
| | 168.00) | 168.00) | 168.00) | 168.00) | 168.00) | 336.00) | 336.00) | 168.00) | 168.00) |
| AUC_{(168 h)}, ng · h/mL | 984 | 6,564 | 8,610 | 16,170 | 32,011 | 9,064 | 10,767 | 15,911 | 35,041 |
| | | (2,695) | (3,847) | (8,720) | (13,049) | (1,981) | (5,370) | (7,231) | (9,722) |
| AUC_{(336 h)}, ng · h/mL | — | — | — | — | — | 26,623 | 26,864 | 31,996 | 70,155 |
| | | | | | | (16,174) | (13,997) | (11,892) | (19,629) |
| C_{max, dn}, ng/mL/mg | 24.2 | 51.7 | 44.1 | 44.6 | 41.5 | 49.0 | 31.7 | 30.8 | 52.5 |
| | | (20.6) | (19.4) | (25.8) | (21.6) | (25.7) | (15.9) | (12.8) | (19.3) |
| AUC_{(168 h, dn)}, | 3,279 | 6,564 | 5,740 | 5,390 | 5,335 | 4,532 | 3,589 | 3,978 | 5,840 |
| ng · h/mL/mg | | (2,695) | (2,565) | (2,907) | (2,175) | (990) | (1,790) | (1,808) | (1,620) |
| AUC_{(336 h, dn)}, | — | — | — | — | — | 13,312 | 8,955 | 7,999 | 11,692 |
| ng · h/mL/mg | | | | | | (8,087) | (4,666) | (2,973) | (3,272) |

Abbreviations: AUC(168 h) = area under the serum concentration-time curve from time 0 to 168 hours; AUC(336 h) = area under the serum concentration-time curve from time 0 to 336 hours; Cmax = maximum serum concentration; dn = dose normalized to 1 mg; n = number of subjects; QXW = every X weeks; SC = subcutaneous; SD = standard deviation; Tmax = time to reach maximum concentration.
Mean ± SD are presented if evaluable subjects >3, and only mean values are presented if evaluable subjects <3. Subjects with a missing concentration around the expected Cmax were excluded from the descriptive statistics.
^{a}n = 4 for AUC(168 h) and AUC(168 h, dn).
^{b}n = 9 for AUC(168 h), AUC(336 h), AUC(168 h, dn) and AUC(336 h, dn).
^{c}n = 9 for AUC(336 h) and AUC(336 h, dn).
^{d}n = 4 for AUC(168 h), AUC(336 h), AUC(168 h, dn) and AUC(336 h, dn).

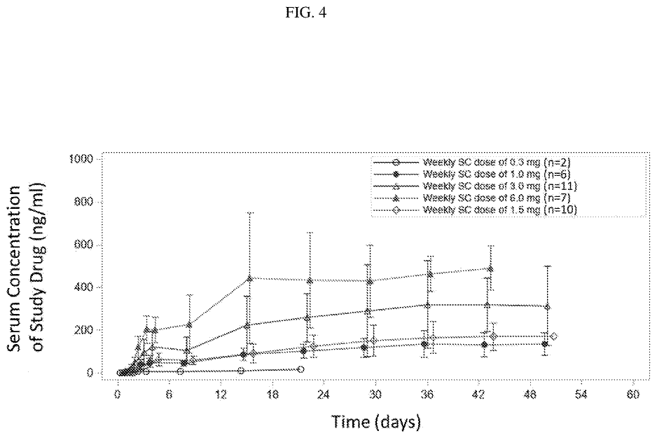

Following multiple SC injections of the study drug, steady state was achieved following the seventh SC injection with Q1W dosing and fourth SC injection with Q2W dosing (FIG. 4 and FIG. 5, respectively). At steady state, serum trough concentration (C_{trough}) increased in an approximately dose-proportional manner following Q1W dosing, while C_{trough} values appeared to be comparable for 2.0 to 4.0 mg Q2W cohorts (Table 8). The mean accumulation ratio for AUC was approximately 4.9 and 1.6 for Q1W dosing and Q2W dosing, respectively.

TABLE 8
Summary of Pharmacokinetic Parameters of the study drug following the Seventh
SC Injection with Q1W Dosing and Fourth SC Injection with Q2W Dosing

| Pharmacokinetics | Weekly (Q1W) | | | | | Once every two weeks (Q2W) | | | |
|---|---|---|---|---|---|---|---|---|---|
| of study drug | 0.3 | 1.0 | 1.5 | 3.0 | 6.0 | 2.0 | 3.0 | 4.0 | 6.0 |
| (mean [SD]) | mg | mg | mg | mg | mg | mg | mg | mg | mg |
| N | 1 | 6 | 6^{a} | 8^{b} | 3 | 4^{c} | 8 | 9^{d} | 1 |
| C_{trough}, ng/mL | 16.3 | 133 | 171 | 340 | 492 | 142 | 128 | 84.0 | 493 |
| | | (56.7) | (63.8) | (118) | (102) | (151) | (83.8) | (55.2) | |
| C_{trough, dn}, | 54.3 | 133 | 114 | 113 | 81.9 | 70.8 | 42.7 | 21.0 | 82.2 |
| ng/mL/mg | | (56.7) | (42.5) | (39.2) | (17.1) | (75.7) | (27.9) | (13.8) | |
| AR_{AUC} | — | — | 3.28 | 5.78 | — | 1.90 | — | 1.48 | — |

Abbreviations: AR_{AUC} = the accumulation ratio; C_{trough} = serum trough concentration; dₙ = dose normalized to 1 mg; n = number of subjects; QXW = every X weeks; SC = subcutaneous; SD = standard deviation.
C_{trough} is the observed concentration just prior to the beginning of a dosing interval.
Mean ± SD are presented if evaluable subjects >3, and only mean values are presented if evaluable subjects <3.
^{a}n = 1 for AR_{AUC}.
^{b}n = 2 for AR_{AUC}.
^{c}n = 1 for AR_{AUC}.
^{d}n = 2 for AR_{AUC}

### Effect of Body Weight on Pharmacokinetics

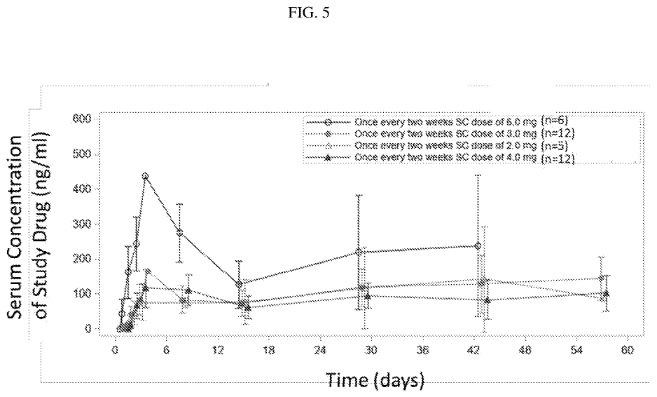

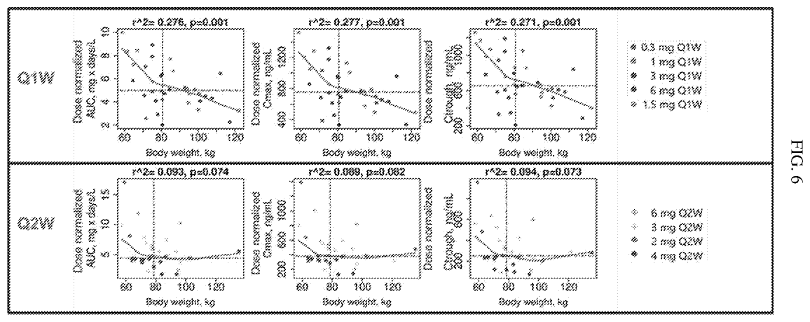

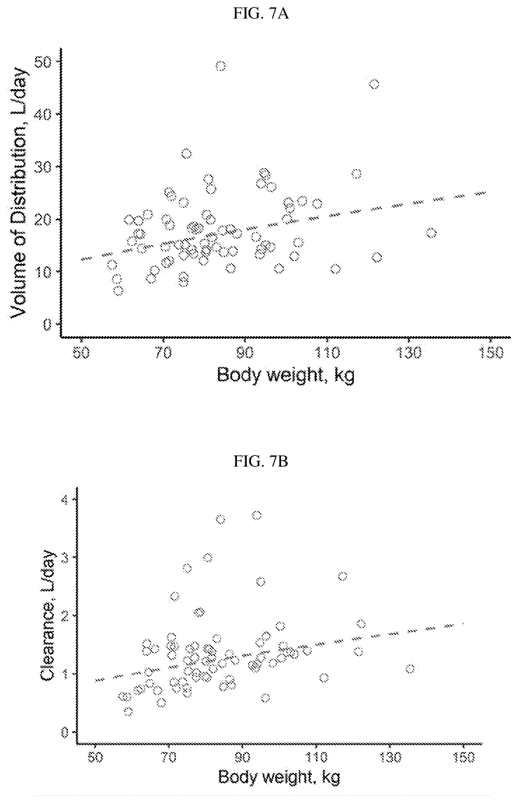

The effect of body weight on pharmacokinetics is shown in FIG. 6 and FIG. 7A and FIG. 7B. Subjects with low body weight are likely to have lower Volume of distribution and Clearance, resulting in higher PK exposures when compared to subjects with high body weight.

### Efficacy

### Efficacy/Pharmacodynamics

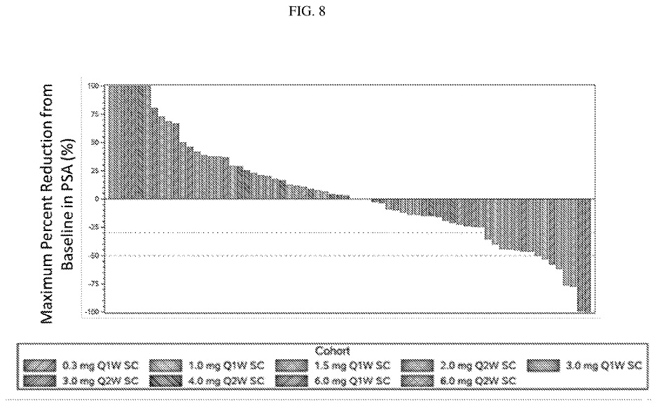

Preliminary PD data for 73 subjects, 38 in the Q1W SC dosing cohorts and 35 in the Q2W SC dosing cohorts were analysed. Maximum prostate-specific antigen (PSA) reductions of at least 50% were reported for 8 subjects: 6 subjects in the Q1W SC dosing cohorts and 2 subjects in the Q2W SC dosing cohorts (FIG. 8). An additional 7 subjects achieved a maximum PSA reduction of at least 30%, of whom 4 subjects were dosed Q1W and 3 subjects dosed Q2W.

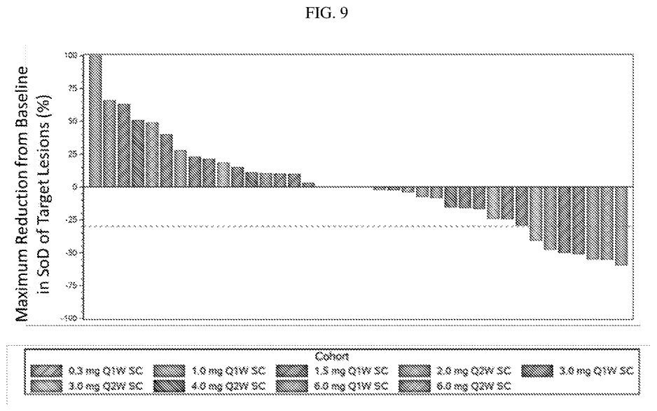

Of the 38 subjects for whom data are available, the maximum percent reduction in the sum of diameters of target lesions per RECIST 1.1 was at least 30% (Partial response or better) for 7 subjects (FIG. 9); 5 subjects from the Q1W SC dosing cohorts and 2 subjects from the Q2W SC dosing cohorts. Confirmed partial responses were observed in 5 patients.

### Summary of Preliminary Efficacy Data

Preliminary efficacy data (PSA and RECIST response) are summarized in Table 9.

TABLE 9
Preliminary efficacy observed across dose level examined.

| | Q1W | | | | | Q2W | | | |
|---|---|---|---|---|---|---|---|---|---|
| | 0.3 | 1 | 1.5 | 3 | 6 | 2 | 3 | 4 | Total |
| | mg | mg | mg | mg | mg** | mg | mg | mg | 73 |
| N | 2 | 6 | 12 | 11 | 7 | 5 | 12 | 12 | |
| RECIST | | | | | | | | | |
| Response | | | | | | | | | |
| Any PR | 0 | 2* | 1 | 1 | 1 | 1 | 1 | 0 | 7 |
| | | (33.3%) | (8.3%) | (9.1%) | (14.3%) | (20.0%) | (8.3%) | | (9.6%) |
| cPR | 0 | 2* | 1 | 0 | 1 | 0 | 1 | 0 | 5 |
| | | (33.3%) | (8.3%) | | (14.3%) | | (8.3%) | | (6.8%) |
| PSA50 | 0 | 1 | 1 | 2 | 2 | 0 | 1 (8.3%) | 0 | 8 |
| | | (16.7%) | (8.3%) | (18.2%) | (28.6%) | | | | (11.0%) |

*Both patients had intra-patient dose escalation to 3 mg QW; one prior to confirmed PR and one after.
**1 patient had uPR prior to dose reduction to 3 mg Q2W due to AEs (day 127). PR was confirmed during the subsequent scan (day 175). The same patient had PSA50 prior to dose reduction; another patient had PSA50 after dose reduction to 3 mg QW due to G3 fall (DLT) on day 16.

### Safety and Tolerability

### Nature and Frequency of Adverse Events

Treatment-emergent AEs were experienced by at least 10% of subjects. Overall, 71 (97.3%) of 73 subjects reported at least 1 TEAE (Table 10 and 11). The most frequently reported TEAEs were fatigue (45.2%), decreased appetite (43.8%), injection site erythema (37.0%), anaemia (32.9%), back pain (24.7%), arthralgia (21.9%), and nausea (19.2%). Incidence of frequent TEAEs is not significantly different across doses.

TABLE 10
Safety profile

| | Study drug Q1W SC dosing | | | | | |
|---|---|---|---|---|---|---|
| TEAEs (≥10%) | 0.3 mg | 1 mg | 1.5 mg | 3 mg | 6 mg | Total |
| n (%) | (n = 2) | (n = 6) | (n = 12) | (n = 11) | (n = 7) | (n = 38) |
| Fatigue | 0 | 2 (33.3%) | 4 (33.3%) | 7 (63.6%) | 4 (57.1%) | 17 (45%) |
| Decreased | 0 | 2 (33.3%) | 3 (25.0%) | 6 (54.5%) | 4 (57.1%) | 15 (40%) |
| appetite | | | | | | |
| Injection site | 0 | 2 (33.3%) | 5 (41.7%) | 5 (45.5%) | 0 | 12 (32%) |
| erythema | | | | | | |
| Anaemia | 1 (50.0%) | 2 (33.3%) | 2 (16.7%) | 4 (36.4%) | 4 (57.1%) | 13 (34%) |
| Back pain | 1 (50.0%) | 3 (50.0%) | 2 (16.7%) | 1 (9.1%) | 2 (28.6%) | 9 (24%) |
| Arthralgia | 0 | 2 (33.3%) | 1 (8.3%) | 3 (27.3%) | 1 (14.3%) | 7 (18%) |
| Nausea | 0 | 1 (16.7%) | 0 | 5 (45.5%) | 3 (42.9%) | 9 (24%) |
| Constipation | 1 (50.0%) | 2 (33.3%) | 1 (8.3%) | 3 (27.3%) | 1 (14.3%) | 8 (21%) |
| Injection site | 0 | 0 | 2 (16.7%) | 4 (36.4%) | 1 (14.3%) | 7 (18%) |
| pruritus | | | | | | |
| Pyrexia | 0 | 0 | 0 | 4 (36.4%) | 3 (42.9%) | 2 (17%) |
| Weight | 0 | 2 (33.3%) | 0 | 3 (27.3%) | 3 (42.9%) | 8 (21%) |
| decreased | | | | | | |
| Vomiting | 0 | 2 (33.3%) | 0 | 3 (27.3%) | 1 (14.3%) | 6 (16%) |
| Erythema | 0 | 0 | 2 (16.7%) | 2 (18.2%) | 3 (42.9%) | 7 (18%) |
| Pruritus | 0 | 2 (33.3%) | 2 (16.7%) | 2 (18.2%) | 0 | 6 (16%) |
| Asthenia | 0 | 0 | 1 (8.3%) | 3 (27.3%) | 2 (28.6%) | 6 (16%) |
| AST increase | 0 | 0 | 2 (16.7%) | 1 (9.1%) | 1 (14.3%) | 4 (11%) |
| Coronavirus | 0 | 0 | 2 (16.7%) | 2 (18.2%) | 0 | 4 (11%) |
| infection | | | | | | |
| Hypotension | 0 | 0 | 2 (16.7%) | 0 | 1 (14.3%) | 3 (8%) |
| Abdominal pain | 0 | 2 (33.3%) | 1 (8.3%) | 3 (27.3%) | 0 | 6 (16%) |

AE, adverse event; AST, aspartate aminotransferase; COVID, coronavirus infectious disease; Q1W, once every week; Q2W, once every two weeks; SC, subcutaneous, TEAE, treatment-emergent adverse event.

TABLE 11
Safety profile

| | Study drug Q2W SC dosing | | | | |
|---|---|---|---|---|---|
| TEAEs (≥10%) | 2 mg | 3 mg | 4 mg | 6 mg | Total |
| n (%) | (n = 5) | (n = 12) | (n = 12) | (n = 6) | (n = 35) |
| Fatigue | 3 (60.0%) | 5 (41.7%) | 4 (33.3%) | 4 (66.7%) | 16 (46%) |
| Decreased appetite | 3 (60.0%) | 5 (41.7%) | 5 (41.7%) | 4 (66.7%) | 17 (49%) |
| Injection site | 0 | 6 (50.0%) | 6 (50.0%) | 3 (50.0%) | 15 (43%) |
| erythema | | | | | |
| Anaemia | 3 (60.0%) | 2 (16.7%) | 4 (33.3%) | 2 (33.3%) | 11 (31%) |
| Back pain | 1 (20.0%) | 4 (33.3%) | 4 (33.3%) | 0 | 9 (26%) |
| Arthralgia | 1 (20.0%) | 4 (33.3%) | 3 (25.0%) | 1 (16.7%) | 9 (26%) |
| Nausea | 1 (20.0%) | 0 | 2 (16.7%) | 2 (33.3%) | 5 (14%) |
| Constipation | 1 (20.0%) | 2 (16.7%) | 1 (8.3%) | 1 (16.7%) | 5 (14%) |
| Injection site | 0 | 2 (16.7%) | 3 (25.0%) | 0 | 5 (14%) |
| pruritus | | | | | |
| Pyrexia | 0 | 1 (8.3%) | 2 (16.7%) | 2 (33.3%) | 5 (14%) |
| Weight decreased | 1 (20.0%) | 2 (16.7%) | 0 | 1 (16.7%) | 4 (11%) |
| Vomiting | 1 (20.0%) | 0 | 3 (25.0%) | 1 (16.7%) | 5 (14%) |
| Erythema | 2 (40.0%) | 1 (8.3%) | 1 (8.3%) | 0 | 4 (11%) |
| Pruritus | 0 | 2 (16.7%) | 1 (8.3%) | 1 (16.7%) | 4 (11%) |
| Asthenia | 0 | 0 | 2 (16.7%) | 1 (16.7%) | 3 (9%) |
| AST increase | 0 | 1 (8.3%) | 2 (16.7%) | 1 (16.7%) | 4 (11%) |
| Coronavirus | 2 (40.0%) | 1 (8.3%) | 1 (8.3%) | 0 | 4 (11%) |
| infection | | | | | |
| Hypotension | 0 | 2 (16.7%) | 2 (16.7%) | 1 (16.7%) | 5 (14%) |
| Abdominal pain | 0 | 1 (8.3%) | 0 | 1 (16.7%) | 2 (6%) |

AE, adverse event;
AST, aspartate aminotransferase;
COVID, coronavirus infectious disease;
Q1W, once every week;
Q2W, once every two weeks;
SC, subcutaneous,
TEAE, treatment-emergent adverse event.

### Treatment-Emergent Serious Adverse Events (SAEs)

Overall, treatment-emergent serious adverse events (SAEs) were reported for 31 (42.5%) subjects in the clinical trial study (17 subjects in Q1W SC dosing cohorts and 14 subjects in the Q2W SC dosing cohorts) (Table 12). The SAEs that were considered related to the study drug by investigators are described as follows. In the 1.0 mg and 3.0 mg study drug Q1W SC dosing cohorts, 1 subject each experienced an SAE of Grade 2 vomiting. In the 1.5 mg study drug Q1W SC dosing cohort, 1 subject experienced an SAE of Grade 2 CRS. In the 6.0 mg study drug Q1W SC dosing cohort, 1 subject experienced an SAE of Grade 3 asthenia and 1 subject experienced SAEs of Grade 2 balance disorder, Grade 2 confusional state, and Grade 3 fall (the TEAE of fall occurred after the dose of the study drug was reduced to 3.0 mg). While treatment with study drug was interrupted, the subject also experienced an SAE of Grade 3 orthostatic hypotension. In the 4.0 mg Q2W SC cohort, 1 subject experienced an SAE of Grade 3 aspartate aminotransferase (AST) increased and in the 6.0 mg study drug Q2W SC cohort, 1 subject experienced SAEs of Grade 2 pyrexia and Grade 2 confusional state and 1 subject experienced an SAE of Grade 1 pyrexia.

### Dose-Limiting Toxicities

2 subjects experienced a total of 3 DLTs in the Study. One subject in the 6.0 mg study drug Q1W SC cohort experienced a DLT of Grade 3 fall and 1 subject in the 6.0 mg study drug Q2W SC cohort experienced a DLT of Grade 3 orthostatic hypotension. At the time of the report, the TEAE of orthostatic hypotension was reported as not recovered/not resolved and study treatment had been withdrawn. Ten days later, the same subject experienced a DLT of Grade 3 syncope that was recovered/resolved.

### Grade 3 or Higher Treatment-Emergent Adverse Events

Grade 3 or higher TEAEs were reported for 40 (54.8%) subjects in the Study. Grade 3 or higher TEAEs (≥5% incidence) reported for more than 1 subject were anaemia (13 subjects, 18%), fatigue (8 subjects 11%), lymphopenia (5 subjects, 7%), asthenia (4 subjects, 6%) and hypertension (4 subjects, 6%), back pain (3 subjects), and arthralgia, AST increased, atrial fibrillation, decreased appetite, dizziness, hypotension, muscular weakness, orthostatic hypotension, pelvic pain, spinal cord compression, and syncope (2 subjects each).

Treatment discontinuation occurred in 7 patients experiencing at least 1 TEAE (blood creatine increased [n=1], weight decrease [n=1], arthralgia [n=1], back pain [n=1], orthostatic hypotension [n=2], hypotension [n=1], pulmonary edema [n=1]. Of 7 patients, 2 patients discontinued study due to treatment-related AES (both with orthostatic hypertension).

Cytokine release syndrome occurred in 4 (6%) of 73 patients and were considered Grade 1 or 2.

Six deaths occurred during the study (progressive disease [n=4]; pulmonary edema [n=1] and COVID-19 [n=1]) and all were considered unrelated to the study drug. Dose limited cytotoxicity was reported in 2 patients (all Grade 3; fall-required hospitalization [n=1]; orthostatic hypotension and syncope [n=1]).

In conclusion, the study drug exhibits a tolerable safety profile at certain doses in mCRPC patients with PSA50 and RECIST responses.

1. A method of treating or slowing the progression of cancer in a subject, the method comprising administering to the subject at least one dose of an anti TMEFF2×CD3 bispecific antibody, wherein the administration is subcutaneous.

2. The method of claim 1 wherein the subcutaneous dose is between about 0.3 mg to about 6 mg of the bispecific antibody.

3. The method of claim 2 wherein the subcutaneous dose is administered once a week or once every two weeks at a dose of about 1.5 mg.

4. The method of claim 1 wherein the cancer is metastatic castration-resistant prostate cancer (mCRPC).

5. The method of claim 4, wherein the anti TMEFF2×CD3 bispecific antibody comprises a first binding domain that binds TMEFF2 and a second binding domain that binds CD3, wherein
a. the first binding domain that binds TMEFF2 comprises the HCDRs of the VH having the amino acid sequence of SEQ ID NO: 13, and the LCDRs of the VL having the amino acid sequence of SEQ ID NO: 14, and the second binding domain that binds CD3 comprises the HCDRs of the VH having the amino acid sequence of SEQ ID NO: 17, and the LCDRs of the VL having the amino acid sequence of SEQ ID NO: 18;
b. the first binding domain that binds TMEFF2 comprises a HCDR1, HCDR2, HCDR3, LCDR1, LCDR2 and LCDR3 of SEQ ID NO 1, 2, 3, 4, 5 and 6, respectively, and the second binding domain that binds CD3 comprises a HCDR1, HCDR2, HCDR3, LCDR1, LCDR2 and LCDR3 of SEQ ID NO 7, 8, 9, 10, 11 and 12, respectively;
c. the first binding domain that binds TMEFF2 comprises a VH and VL of SEQ ID NO: 13 and 14, respectively, and the second binding domain that binds CD3 comprises a VH and VL of SEQ ID NO: 17 and 18, respectively; and/or
d. the first binding domain that binds TMEFF2 comprises a HC1 and LC1 of SEQ ID NO: 15 and 16 respectively; and the second binding domain that binds CD3 comprises a HC2 and LC2 of SEQ ID NO: 19 and 20 respectively


SEQUENCE LISTING (29 sequences)

SEQ ID NO: 1 - protein, 5 aa, synthetic construct
SYSMS

SEQ ID NO: 2 - protein, 17 aa, synthetic construct
VISGSGGFTDYADSVKG

SEQ ID NO: 3 - protein, 9 aa, synthetic construct
MPLNSPHDY

SEQ ID NO: 4 - protein, 11 aa, synthetic construct
RASQGIRNDLG

SEQ ID NO: 5 - protein, 7 aa, synthetic construct
AASSLQS

SEQ ID NO: 6 - protein, 9 aa, synthetic construct
LQDYNYPLT

SEQ ID NO: 7 - protein, 7 aa, synthetic construct
NNNAAWS

SEQ ID NO: 8 - protein, 18 aa, synthetic construct
RTYYRSKWLYDYAVSVKS

SEQ ID NO: 9 - protein, 8 aa, synthetic construct
GYSSSFDY

SEQ ID NO: 10 - protein, 14 aa, unidentified
TGTSSNIGTYKFVS

SEQ ID NO: 11 - protein, 7 aa, synthetic construct
EVSKRPS

SEQ ID NO: 12 - protein, 10 aa, synthetic construct
VSYAGSGTLL

SEQ ID NO: 13 - protein, 118 aa, synthetic construct
EVQLLESGGGLVQPGGSLRLSCAASGFTFSSYSMSWVRQAPGKGLEWVSVISGSGGFTDYADSVKGRFTISRDNSKNTLYLQMNSLRAEDTAVYYCARMPLNSPHDYWGQGTLVTVSS

SEQ ID NO: 14 - protein, 107 aa, synthetic construct
DIQMTQSPSSLSASVGDRVTITCRASQGIRNDLGWYQQKPGKAPKLLIYAASSLQSGVPSRFSGSGSGTDFTLTISSLQPEDFATYYCLQDYNYPLTFGGGTKVEIK

SEQ ID NO: 15 - protein, 445 aa, synthetic construct
EVQLLESGGGLVQPGGSLRLSCAASGFTFSSYSMSWVRQAPGKGLEWVSVISGSGGFTDYADSVKGRFTISRDNSKNTLYLQMNSLRAEDTAVYYCARMPLNSPHDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 214 aa, synthetic construct
DIQMTQSPSSLSASVGDRVTITCRASQGIRNDLGWYQQKPGKAPKLLIYAASSLQSGVPSRFSGSGSGTDFTLTISSLQPEDFATYYCLQDYNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 120 aa, synthetic construct
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 18 - protein, 110 aa, synthetic construct
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 19 - protein, 447 aa, synthetic construct
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 20 - protein, 216 aa, synthetic construct
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 91 aa, Homo sapiens
NTTTTTKSEDGHYARTDYAENANKLEESAREHHIPCPEHYNGFCMHGKCEHSINMQEPSCRCDAGYTGQHCEKKDYSVLYVVPGPVRFQYV

SEQ ID NO: 22 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 23 - DNA, 1335 bp, synthetic construct
  source 1..1335: mol_type = other DNA
gaggtgcagctgctggaaagcggcggaggcctggtgcagcccggaggaagcctgagactcagctgtgccgccagcggcttcaccttcagcagctacagcatgagctgggtcaggcaggcccctggcaaaggactggagtgggtgagcgtgattagcggcagcggcggcttcaccgattacgccgacagcgtgaagggcaggttcaccatcagcagggacaatagcaagaacaccctgtacctgcagatgaacagcctgagggccgaggacaccgccgtgtactactgcgccaggatgcccctgaacagccctcacgactactggggccagggaaccctggtgaccgtgtccagcgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaaaacctacacttgcaacgtagatcacaagcccagcaacaccaaggtggacaagagagttgagtccaaatatggtcccccatgcccaccatgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcagatggcaggaggggaatgtcttctcatgctccgtgatgcatgaggctctgcacaaccactacacacagaagagcctctccctgtctctgggtaaa

SEQ ID NO: 24 - DNA, 645 bp, synthetic construct
  source 1..645: mol_type = other DNA
gacatccagatgacccagagccctagcagcctgagcgctagcgtgggcgacagggtgaccatcacctgcagggccagccagggcatcagaaacgacctgggctggtaccagcagaagcccggcaaggcccccaagctgctgatctacgccgccagcagcctgcagagcggagtgcctagcaggttcagcggaagcggcagcggcaccgacttcaccctgaccatcagcagcctgcagcccgaggacttcgccacctactactgcctgcaggactacaactaccccctgacattcggcggcggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 25 - DNA, 1341 bp, synthetic construct
  source 1..1341: mol_type = other DNA
caggtgcagctgcagcagtctggccctagactcgtgcggccttcccagaccctgtctctgacctgtgccatctccggcgactccgtgttcaacaacaacgccgcctggtcctggatccggcagagcccttctagaggcctggaatggctgggccggacctactaccggtccaagtggctgtacgactacgccgtgtccgtgaagtcccggatcaccgtgaaccctgacacctcccggaaccagttcaccctgcagctgaactccgtgacccctgaggacaccgccctgtactactgcgccagaggctactcctcctccttcgactattggggccagggcaccctcgtgaccgtgtcctctgcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaaaacctacacctgcaacgtagatcacaagcccagcaacaccaaggtggacaagagagttgagtccaaatatggtcccccatgcccaccatgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcctcctctacagcaagctaaccgtggacaagagcaggtggcaggaggggaatgtcttctcatgctccgtgatgcatgaggctctgcacaaccactacacacagaagagcctctccctgtctctgggtaaa

SEQ ID NO: 26 - DNA, 648 bp, synthetic construct
  source 1..648: mol_type = other DNA
cagtctgctctgacccagcctgcctccgtgtctggctctcccggccagtccatcaccatcagctgtaccggcacctcctccaacatcggcacctacaagttcgtgtcctggtatcagcagcaccccgacaaggcccccaaagtgctgctgtacgaggtgtccaagcggccctctggcgtgtcctccagattctccggctccaagtctggcaacaccgcctccctgaccatcagcggactgcaggctgaggaccaggccgactaccactgtgtgtcctacgctggctctggcaccctgctgtttggcggaggcaccaagctgaccgtgctgggtcagcccaaggctgcacccagtgtcactctgttcccgccctcctctgaggagcttcaagccaacaaggccacactggtgtgtctcataagtgacttctacccgggagccgtgacagtggcctggaaggccgatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 27 - protein, 374 aa, Homo sapiens
MVLWESPRQCSSWTLCEGFCWLLLLPVMLLIVARPVKLAAFPTSLSDCQTPTGWNCSGYDDRENDLFLCDTNTCKFDGECLRIGDTVTCVCQFKCNNDYVPVCGSNGESYQNECYLRQAACKQQSEILVVSEGSCATDAGSGSGDGVHEGSGETSQKETSTCDICQFGAECDEDAEDVWCVCNIDCSQTNFNPLCASDGKSYDNACQIKEASCQKQEKIEVMSLGRCQDNTTTTTKSEDGHYARTDYAENANKLEESAREHHIPCPEHYNGFCMHGKCEHSINMQEPSCRCDAGYTGQHCEKKDYSVLYVVPGPVRFQYVLIAAVIGTIQIAVICVVVLCITRKCPRSNRIHRQKQNTGHYSSDNTTRASTRLI

SEQ ID NO: 28 - protein, 280 aa, Homo sapiens
FPTSLSDCQTPTGWNCSGYDDRENDLFLCDTNTCKFDGECLRIGDTVTCVCQFKCNNDYVPVCGSNGESYQNECYLRQAACKQQSEILVVSEGSCATDAGSGSGDGVHEGSGETSQKETSTCDICQFGAECDEDAEDVWCVCNIDCSQTNFNPLCASDGKSYDNACQIKEASCQKQEKIEVMSLGRCQDNTTTTTKSEDGHYARTDYAENANKLEESAREHHIPCPEHYNGFCMHGKCEHSINMQEPSCRCDAGYTGQHCEKKDYSVLYVVPGPVRFQYV

SEQ ID NO: 29 - protein, 41 aa, Homo sapiens
HHIPCPEHYNGFCMHGKCEHSINMQEPSCRCDAGYTGQHCE